SEQUENCE LISTING (22 sequences)

SEQ ID NO: 1 - DNA, 19 bp, Artificial Sequence
  Primer forward for CD44
ctgccgctttgcaggtgta

SEQ ID NO: 2 - DNA, 21 bp, Artificial Sequence
  Primer reverse for CD44
cattgtgggcaaggtgctatt

SEQ ID NO: 3 - DNA, 21 bp, Artificial Sequence
  Primer forward for CD73
gcctgggagcttacgattttg

SEQ ID NO: 4 - DNA, 20 bp, Artificial Sequence
  Primer reverse for CD73
tagtgccctggtactggtcg

SEQ ID NO: 5 - DNA, 21 bp, Artificial Sequence
  Primer forward for CD90
atcgctctcctgctaacagtc

SEQ ID NO: 6 - DNA, 21 bp, Artificial Sequence
  Primer reverse for CD90
ctcgtactggatgggtgaact

SEQ ID NO: 7 - DNA, 21 bp, Artificial Sequence
  Primer forward for CD105
tgcacttggcctacaattcca

SEQ ID NO: 8 - DNA, 20 bp, Artificial Sequence
  Primer reverse for CD105
agctgcccactcaaggatct

SEQ ID NO: 9 - DNA, 23 bp, Artificial Sequence
  Primer forward for CD34
ctacaacacctagtacccttgga

SEQ ID NO: 10 - DNA, 22 bp, Artificial Sequence
  Primer reverse for CD34
ggtgaacactgtgctgattaca

SEQ ID NO: 11 - DNA, 23 bp, Artificial Sequence
  Primer forward for CD45
accacaagtttactaacgcaagt

SEQ ID NO: 12 - DNA, 22 bp, Artificial Sequence
  Primer reverse for CD45
tttgagggggattccaggtaat

SEQ ID NO: 13 - DNA, 21 bp, Artificial Sequence
  Primer forward for GAPDH
ggagcgagatccctccaaaat

SEQ ID NO: 14 - DNA, 23 bp, Artificial Sequence
  Primer reverse for GAPDH
ggctgttgtcatacttctcatgg

SEQ ID NO: 15 - DNA, 21 bp, Artificial Sequence
  Primer forward for SPP1
ctccattgactcgaacgactc

SEQ ID NO: 16 - DNA, 23 bp, Artificial Sequence
  Primer reverse for SPP1
caggtctgcgaaacttcttagat

SEQ ID NO: 17 - DNA, 19 bp, Artificial Sequence
  Primer forward for SOX9
agcgaacgcacatcaagac

SEQ ID NO: 18 - DNA, 20 bp, Artificial Sequence
  Primer reverse for SOX9
ctgtaggcgatctgttgggg

SEQ ID NO: 19 - DNA, 19 bp, Artificial Sequence
  Primer forward for MAP2
cactggcggtgcaacaaga

SEQ ID NO: 20 - DNA, 23 bp, Artificial Sequence
  Primer reverse for MAP2
tttcataacagcggaggcatttc

SEQ ID NO: 21 - DNA, 19 bp, Artificial Sequence
  Primer forward for PPARG
gctggacgtcctggtgaag

SEQ ID NO: 22 - DNA, 23 bp, Artificial Sequence
  Primer reverse for PPARG
acgttgtccagcaataccctgag

(19) United States
(12) Patent Application Publication
(10) Pub. No.: US 2017/0204366 A1
(43) Pub. Date: Jul. 20, 2017

(54) CULTURE SUBSTRATE

(71) Applicant: AISIN SEIKI KABUSHIKI KAISHA, Kariya-shi (JP)

(72) Inventors: Ryuichiro SASAKI, Kariya-shi (JP); Toshihiro ASO, Nagakute-shi (JP); Takao HANAWA, Nakano-ku (JP); Peng CHEN, Tsukuba-shi (JP)

(73) Assignee: AISIN SEIKI KABUSHIKI KAISHA, Kariya-shi (JP)

(21) Appl. No.: 15/265,070

(22) Filed: Sep. 14, 2016

(30) Foreign Application Priority Data
Sep. 14, 2015 (JP) 2015-181147
Sep. 13, 2016 (JP) 2016-178263

(51) Int. Cl. C12N 5/00

(52) U.S. Cl. CPC C12N 5/0068; C12N 2535/10; C12N 2533/00

(57) ABSTRACT

Provided is a culture substrate having a periodic fine structure in the order of micrometers and a periodic fine structure in the order of nanometers on the same surface where stem cells are to be cultured on the surface.

### CROSS REFERENCE TO RELATED APPLICATION

The present application is based on Japanese Patent Application No. 2015-181147, which was filed on Sep. 14, 2015, and Japanese Patent Application No. 2016-178263, which was filed on Sep. 13, 2016, and claims priority from these Japanese Patent Applications under 35 U.S.C. §119. These Japanese Patent Applications are incorporated herein by reference.

### TECHNICAL FIELD

The present disclosure relates to a culture substrate, and particularly to a culture substrate having a periodic fine structure in the order of micrometers and a periodic fine structure in the order of nanometers on the same surface where stem cells are to be cultured on the surface, and a method for producing a culture substrate.

### RELATED ART

In recent years, regenerative medicine has been studied actively and is expected to play a part in conventional medical treatment. Regenerative medicine is treatment for regenerating tissue and viscera that have a functional disorder or a dysfunction caused by a disease or injury to restore their functions. Stem cells play an important role in achieving regenerative medicine. Stem cells have the ability to self replicate and the ability to differentiate, and this ability of stem cells to differentiate can be used to artificially produce tissue and viscera necessary for curing diseases and injuries.

Stem cells can also be used in a drug development screening in which, for example, biological cells obtained by inducing the differentiation of stem cells are used to evaluate the pharmacology and toxicity of a development candidate drug; the clarification of a development mechanism, a differentiation mechanism, and a disease mechanism; techniques for producing useful substances such as proteins having biological functions that can be used as protein drugs; and the like. Therefore, techniques using stem cells are expected to be applied to a broad technical field including medical treatment, drug development, and the like.

However, the proliferation and differentiation of stem cells need to be appropriately controlled in order to regenerate tissue and viscera that can be used in transplant treatment, a drug development screening, and the like. That is, in order to apply stem cells to regenerative medicine, it is necessary to solve some problems in that, for example, a culture technique is established with which stem cells can be safely and stably cultured and proliferated while maintaining undifferentiation properties, and the differentiation into a desired type of cell can be efficiently induced.

For example, JP 2014-82956A (Reference 1) states that N-cadherin is a differentiation controlling factor in controlling the differentiation of pluripotent stem cells into nerve cells and plays an important role in the development of the nervous system. It has been confirmed that pluripotent stem cells selectively differentiate into nerve cells by immobilizing the N-cadherin or its homologue on the surface of a culture substrate. JP 2013-223446A (Reference 2) states that an insulin-like growth factor binding protein (abbreviated as “IGFBP” hereinafter) is a differentiation controlling factor in controlling the differentiation of pluripotent stem cells into cardiac muscle cells and strongly promotes the induction into cardiac muscle cells. It has been confirmed that pluripotent stem cells selectively differentiate into cardiac muscle cells by immobilizing the IGFBP or its homologue on the surface of a culture substrate.

However, both the differentiation controlling factors stated in Reference 1 and Reference 2 are organic substances having high biochemical activity. Therefore, culture substrates on which these differentiation controlling factors have been immobilized need to be stored in a sterilized environment until they are to be used, for example, and thus advanced knowledge, techniques, and devices are required.

Culturing of stem cells has problems incidental to feeder cells. The culturing of stem cells has been generally performed as coculturing with feeder cells. Feeder cells provide factors that are necessary for the survival, proliferation, and undifferentiation property maintenance of stem cells as well as scaffolds for cell adhesion. However, coculturing with feeder cells causes mixing of components derived from feeder cells, and therefore, a safety problem in biological application such as regenerative medicine arises. In addition, it is not easy to stably provide high-quality feeder cells. The technique disclosed in Reference 1 above is a stem cell culturing system without feeder cells, but as mentioned above, advanced knowledge, techniques, and devices are required.

What kind of influences a fine structure of the surface of a culture substrate has on the survival, proliferation, cell division process, undifferentiation property maintenance, and cell adhesion of stem cells during the culturing of stem cells has been examined. JP 2014-138605A (Reference 3) describes a possibility that the surface fine structure causes the differentiation of pluripotent stem cells. With the technique disclosed in Reference 3, topographical projections (having a circular shape, a star shape, a rectangular shape, a crescent shape, or the like) are provided on lattice points on the surface of the culture substrate.

Reference 3 suggests that the intervals between the projections and the cross-sectional diameters of the projections should be 1 to 2 μm and 1 to 8 μm, respectively, as those having influences on the differentiation of stem cells. As a method for producing such projections, nanoimprinting, laser ablation, chemical etching, plasma spray coating, spray grinding, engraving, scratching, and micromachining are suggested in addition to photo lithography, electron-beam lithography, and hot embossing.

However, it is difficult to precisely form the shapes suggested in Reference 3 using the laser ablation in which a light condensing spot generally has a size of several micrometers to several tens of micrometers. Therefore, it is thought that a high-cost processing means such as photolithography needs to be used as a method that can be actually used to produce the aforementioned projections.

JP 2010-227551A (Reference 4) states that titanium is irradiated with a high-intensity femto-second laser pulse to produce surface-processed titanium in which hemispherical protrusions in the order of micrometers with a groove surrounding the protrusion are formed and a fine surface structure including a large number of fine spherical projections and fine recesses in the order of nanometers are formed on the entirety of the surfaces of the surrounding grooves and hemispherical protrusions. Titanium rarely causes an immune response when embedded in a living organism, and thus is the mainstream of implant materials such as artificial joints and artificial dental roots. However, problems to be improved upon remain in that cells poorly adhere to the surface of titanium, which is originally foreign matter for a living organism, and thus it is difficult to regenerate tissue, for example. In contrast, it is reported that, in the technique disclosed in Reference 4, the adhesiveness of osteoblasts to the surface of titanium is improved by performing micromachining on the surface of titanium, thus promoting the proliferation of osteoblastic cells, which are precursor cells of osteoblasts isolated from bone marrow, and inducing the differentiation into osteoblasts.

However, although it has been confirmed that the proliferation of osteoblastic cells and the differentiation of osteoblastic cells into osteoblasts is promoted by the technique disclosed in Reference 4, application to stem cells having a high undifferentiation property has not been examined. Reference 4 states that 2- to 20-μm hemispherical protrusions surrounded by a groove and a surface structure including 100- to 300-nm fine spherical projections and fine recesses are formed by irradiating the surface of titanium with a femto-second laser pulse of 800 μJ. In view of this, the fine structure in the order of nanometers is formed accompanying the formation of the fine structure in the order of micrometers.

### SUMMARY

In this manner, culture substrates that are unlikely to suffer the above-mentioned disadvantages are in demand.

An aspect of the present disclosure is a culture substrate having a periodic fine structure in the order of micrometers and a periodic fine structure in the order of nanometers on the same surface where stem cells are to be cultured on the surface.

### BRIEF DESCRIPTION OF DRAWINGS

The foregoing characteristics and properties of the present disclosure as well as further characteristics and properties thereof will be more apparent from the following specific description provided with reference to the accompanying drawings. Here:

FIG. 1 shows the result from the examination of production of a culture substrate (determination of conditions for laser processing) in Example 2, showing an SEM image of the processed surface of periodic microgrooves.

FIG. 2 shows the result from the examination of production of a culture substrate (determination of conditions for laser processing) in Example 2, showing the observation result of the depth of the periodic microgrooves.

FIG. 3 shows the result from the examination of production of a culture substrate (determination of conditions for laser processing) in Example 2, showing SEM images of the cross sections of the periodic microgrooves.

FIG. 4 shows the result from the examination of production of a culture substrate (determination of conditions for laser processing) in Example 2, showing an SEM image of the processed surface of periodic nanogrooves.

FIG. 5 shows the result from the examination of production of a culture substrate (determination of conditions for laser processing) in Example 2, showing the observation result of the depth of the periodic nanogrooves.

FIG. 6 shows the result from the examination of production of a culture substrate (determination of conditions for laser processing) in Example 2, showing SEM images of the cross sections of the periodic nanogrooves.

FIG. 7 shows the result from the examination of production of a culture substrate (determination of conditions for laser processing) in Example 2, showing an SEM image of the processed surface of hybrid periodic grooves.

FIG. 8 shows the result from the examination of production of a culture substrate (determination of conditions for laser processing) in Example 2, showing the observation result of the depth of the hybrid periodic grooves.

FIG. 9 shows the result from the examination of production of a culture substrate (determination of conditions for laser processing) in Example 2, showing SEM images of the cross sections of the hybrid periodic grooves.

FIG. 10 shows schematic views of culture substrates produced in Example 3 in which biological cell affinity testing (calculation of a cell density) is performed.

FIG. 11 is a graph showing the results from the biological cell affinity testing (calculation of a cell density) performed in Example 3.

FIG. 12 shows the results from biological cell affinity testing (observation of a cell morphology) performed in Example 4, showing fluorescent micrographs illustrating the cell morphology in a case where a culture substrate of a control (mirror surface) is used.

FIG. 13 shows the results from the biological cell affinity testing (observation of a cell morphology) performed in Example 4, showing fluorescent micrographs illustrating the cell morphology in a case where a culture substrate on which the periodic microgrooves are formed is used.

FIG. 14 shows the results from the biological cell affinity testing (observation of a cell morphology) performed in Example 4, showing fluorescent micrographs illustrating the cell morphology in a case where a culture substrate on which the hybrid periodic grooves are formed is used.

FIG. 15 shows the results from biological cell affinity testing (observation of a cell morphology) performed in Example 4, showing fluorescent micrographs illustrating the cell morphology in a case where a culture substrate on which the periodic nanogrooves are formed is used.

FIG. 16 is a graph showing the results from the confirmation of the undifferentiation property of MSCs on a culture substrate performed in Example 5.

FIG. 17 is a graph showing the results from the analysis of the induction of the differentiation of MSCs on a culture substrate (confirmation using differentiation markers) performed in Example 6.

FIG. 18 shows fluorescent micrographs showing the results from the analysis of the induction of the differentiation of MSCs on a culture substrate (confirmation by the observation under a fluorescent microscope) performed in Example 7.

FIG. 19 shows fluorescent micrographs showing the results from the analysis of the induction of the differentiation of MSCs on a culture substrate (confirmation by immunofluorescent staining) performed in Example 8.

FIG. 20 is a graph showing the results from the evaluation of the induction of the differentiation of MSCs into bone (measurement of alkaline phosphatase activity) performed in Example 9.

FIG. 21 is a graph showing the results from the evaluation of the induction of the differentiation of MSCs into bone (evaluation of calcification ability) performed in Example 10.

FIG. 22 shows the result from the examination of production of a culture substrate in Example 11, showing an SEM image of the cross section of periodic lattice microgrooves.

FIG. 23 shows the result from the examination of production of a culture substrate in Example 11, showing an SEM image of the cross sections of hybrid periodic lattice grooves+periodic projections.

FIG. 24 is a graph showing the results from the analysis of the induction of the differentiation of MSCs on a culture substrate (confirmation using differentiation markers) performed in Example 12.

### DETAILED DESCRIPTION

A culture substrate according to an embodiment disclosed here will be explained with reference to the attached drawings.

A periodic fine structure 2 in the order of micrometers and a periodic fine structure 3 in the order of nanometers are simultaneously formed on a surface of a culture substrate 1 of the present disclosure. Hereinafter, the structure formed on the culture substrate of the present disclosure in which the periodic fine structure 2 in the order of micrometers and the periodic fine structure 3 in the order of nanometers coexist may be abbreviated as “hybrid periodic structure”, the periodic fine structure 2 in the order of micrometers alone may be abbreviated as “periodic microstructure”, and the periodic fine structure 3 in the order of nanometers alone may be abbreviated as “periodic nanostructure”.

It is preferable to select a material that is chemically stable and has good biocompatibility and biological cell affinity as a material for the culture substrate 1 of the present disclosure. Here, “chemically stable” means “having necessary strength, durability, or wear resistance”. For example, when used as an implant material, the culture substrate 1 needs to have mechanical biocompatibility depending on the portion in which the culture substrate 1 is to be embedded. “Biocompatiblity” means a property of having no influence on a living organism and components derived from a living organism, such as cells, tissue, viscera, and blood as well as not being affected by the living organism and the components derived from a living organism, and being hardly recognized as foreign matter in the living organism. “Biological cell affinity” particularly means a property of having no influence on biological cells and components derived from biological cells as well as not being affected by the biological cells and the components derived from biological cells, and hardly inhibiting the survival, proliferation, and the like of the biological cells. Specifically, having no toxicity, no carcinogenicity, and no antigenicity, giving rise to no blood coagulation, no hemolysis, and no metabolic disorders, and the like are taken as examples of biocompatibility and biological cell affinity. It is preferable to select a material having biocompatibility and biological cell affinity at a required level according to the application of culture substrate 1.

Specifically, known substances such as metal materials, ceramic materials, and synthetic macromolecular materials can be used as long as the substances have the above-mentioned properties. Examples of metal materials include titanium, a titanium alloy, a titanium oxide, stainless steel, niobium, a niobium alloy, a niobium oxide, tantalum, a tantalum alloy, a tantalum oxide, a nickel-chromium alloy, and a chromium-cobalt alloy. “Alloy” refers to a substance that is constituted by a plurality of metal elements or a metal element and a non-metal element and has metallic properties. For example, a substance obtained by mixing titanium with one or more elements other than titanium, such as nickel, niobium, tantalum, molybdenum, zirconium, and platinum, and adjusting the composition can be used as a titanium alloy. Examples of ceramic materials include alumina, an alumina oxide, zirconium, a zirconium oxide, and hydroxyapatite. A substance obtained by adding another additive to a ceramic material and molding a resultant mixture, a substance obtained by coating the surface of the above-mentioned metal material with a molten ceramic material, and, conversely, a substance obtained by coating a ceramic material with a metal material such as an alloy can also be used. Examples of synthetic macromolecular materials include silicone and polyurethane.

There is no limitation on the shape and the size of the culture substrate 1 of the present disclosure. The shape can be selected as appropriate from a plate shape, a cube shape, a pillar shape, a rod shape, a fibrous shape, a spherical shape, a granular shape, and a massive shape according to the application. The periodic fine structure may be formed on all surfaces of the culture substrate 1, some surfaces thereof, or a part of the surface. When the culture substrate 1 of the present disclosure is used as an implant, the shape and the size thereof can be determined as appropriate according to a portion in which the culture substrate 1 is to be embedded or a tissue whose regeneration is desired.

The “periodic fine structure” formed on the surface of the culture substrate 1 of the present disclosure means a structure in which fine recessed portions and line raised portions are periodically provided at constant intervals on the same flat surface of a substrate. Examples of the recessed portions include grooves and holes, and examples of the raised portions include projecting lines and projections.

The grooves and the projecting lines can be formed in a linear shape, a curved shape, a polygonal line shape, or the like, and a plurality of projecting lines or grooves are arranged in parallel, in a concentric shape, in a lattice shape, or in a spiral shape, for example. Examples of the cross-sectional shape of the projecting lines and the grooves in a direction orthogonal to the longitudinal direction include a quadrilateral, a triangle (V shape), and a semicircle (U shape). It is preferable to form a periodic groove structure in which a plurality of linear projecting lines or grooves are successively disposed in parallel at constant intervals. It is also preferable to form a periodic lattice structure in which a plurality of linear projecting lines or grooves that are successively disposed in parallel at constant intervals are combined to be arranged in a grid pattern. Regions surrounded by the projecting lines or the grooves in the periodic lattice structure can be formed in a square, a rectangle, a parallelogram, a rhombus, a triangle, or the like, and there is no limitation on the angle of the intersection portions of the projecting lines or the grooves.

The projections and the holes can be formed in a pyramid shape such as a triangular pyramid, a quadrangular pyramid, or a hexagonal pyramid, a circular cone shape, a circular cylindrical shape, a hemispherical shape, a waveform shape, a bell shape, or the like. The projection can also be referred to as “dot”. A plurality of projections and holes are arranged in parallel, in a concentric shape, in a lattice shape, in a spiral shape, or in a random manner, for example. The cross-sectional areas of the projection and hole in a direction orthogonal to the height direction may change from a bottom portion toward a top portion, or need not change. If they change, they may have a shape in which the cross-sectional area gradually decreases, or a shape in which the cross-sectional area increases, or a shape in which an increase in the cross-sectional area and a decrease in the cross-sectional area are combined.

In a hybrid periodic structure 4 of the culture substrate 1 of the present disclosure, a periodic microstructure 2 and a periodic nanostructure 3 may be formed by combining any shapes as long as the periodic microstructure 2 and the periodic nanostructure 3 are simultaneously formed on the surface of the culture substrate 1. Therefore, both the periodic microstructure 2 and the periodic nanostructure 3 may be formed as periodic projecting lines, periodic grooves, periodic projections, or periodic holes. Alternatively, one of them may be formed as periodic projecting lines or periodic grooves, and the other may be formed as periodic projections or periodic holes.

When both the periodic microstructure 2 and the periodic nanostructure 3 are formed as the periodic projecting lines or the periodic grooves, the periodic nanostructure 3 can be arranged on the periodic microstructure 2 so as to extend in the same direction, or may be arranged so as to extend in a different direction. For example, when the periodic microstructure 2 includes individual elements arranged in parallel to extend in one direction, individual elements of the periodic nanostructure 3 can be arranged in parallel to extend in the same direction. Moreover, the periodic nanostructure 3 may be arranged on the periodic microstructure 2 at a certain angle. At this time, the angle can be determined as appropriate. Here, the hybrid periodic structure 4 in which the periodic microstructure 2 and the periodic nanostructure 3 are formed as parallel grooves may be referred to as “hybrid periodic grooves 4a”.

When the periodic microstructure 2 is formed as the periodic projecting lines or periodic grooves, and the periodic nanostructure 3 is formed as the periodic projections or periodic holes, the periodic nanostructure 3 can be arranged on the periodic microstructure 2 in a longitudinal direction and in a lateral direction. Intervals in the longitudinal direction and the lateral direction may be the same or different. Preferably, it is possible to form the periodic microstructure 2 as periodic lattice grooves 2b and the periodic nanostructure 3 as periodic projections 3b. Here, the hybrid periodic structure 4 in which the periodic microstructure 2 is formed as the periodic lattice grooves 2b and the periodic nanostructure 3 is formed as the periodic projections 3b may be referred to as “hybrid periodic lattice grooves+periodic projections 4b”.

Here, the “periodic fine structure 2 in the order of micrometers” means a periodic fine structure formed to dimensions that are to an extent such that it is appropriate to indicate the dimensions in μm units, and the fine structure formed to dimensions that are to an extent such that a periodic structure can be controlled artificially. Here, this means that when the periodic fine structure is formed as the periodic projecting lines or periodic grooves, they are formed to have widths, heights or depths, and pitches in the order of micrometers. Specifically, this means that they have widths and pitches of 1 to 100 μm and heights or depths of about 0.1 to 10 μm. Moreover, this means that when the periodic fine structure is formed as periodic rectangular projections or periodic rectangular holes, they have side lengths, heights or depths, and pitches in the order of micrometers, and when the periodic fine structure is formed as periodic circular projections or periodic circular holes, they have diameters, heights or depths, pitches, and the like in the order of micrometers. Specifically, this means that they have side lengths or diameters and pitch widths of 1 to 100 μm and heights and depths of about 0.1 to 10 μm.

The “periodic fine structure 3 in the order of nanometers” means a periodic fine structure formed to dimensions that are to an extent such that it is appropriate to indicate the dimensions in nm units, and the fine structure formed to dimensions that are to an extent such that the formation of the structure by laser irradiation or the like can be recognized as a phenomenon. Specifically, the dimensions are 10 to 1000 nm. Here, this means that when the periodic fine structure is formed as periodic projecting lines or periodic grooves, they are formed to have widths, heights or depths, and pitches in the order of nanometers. Moreover, this means that when the periodic fine structure is formed as periodic rectangular projections or periodic rectangular holes, they have side lengths, heights or depths, and pitches in the order of nanometers, and when the periodic fine structure is formed as periodic circular projections or periodic circular holes, they have diameters, heights or depths, pitches, and the like in the order of nanometers.

When the periodic fine structure of the culture substrate 1 of the present disclosure is formed by arranging periodic grooves 2a in the order of micrometers and periodic grooves 3a in the order of nanometers in parallel so as to extend in the same direction, it is preferable to set the groove widths of the periodic grooves 2a, in the order of micrometers, to 1 to 20 μm and particularly 5 to 10 μm, the depths thereof to 0.3 to 2 μm and particularly 0.6 to 1 μm, and the pitches thereof to 1 to 100 μm and particularly 10 to 20 μm, and it is preferable to set the groove widths of the periodic grooves 3a, in the order of nanometers, to 0.1 to 0.5 μm (100 to 500 nm), the depths thereof to 0.01 to 0.5 μm (10 to 500 nm), and the pitches thereof to 0.1 to 1 μm (100 to 1000 nm). Specifically, the groove widths of the periodic grooves 2a in the order of micrometers are set to 6 μm, the depths thereof are set to 0.6 μm or 1 μm, and the pitches thereof are set to 12 μm. Furthermore, the groove widths of the periodic grooves 3a in the order of nanometers are set to 0.3 μm (300 nm), the depths thereof are set to 0.2 μm (200 nm), and the pitches thereof are set to 0.5 to 0.8 μm (500 to 800 nm) and particularly preferably 0.7 μm (700 nm).

When the periodic fine structure of the culture substrate 1 of the present disclosure is formed to include the periodic grooves 2a in the order of micrometers and the periodic rectangular projections 3b or the periodic circular projections 3b in the order of nanometers, it is preferable to set the groove widths of the periodic grooves 2a, in the order of micrometers, to 1 to 20 μm, the depths thereof to 0.3 to 2 μm, and the pitches thereof to 1 to 100 μm, and it is preferable to set the projection diameters of the periodic projections, in the order of nanometers, to 0.1 to 1 μm (100 to 1000 nm), the heights thereof to 0.01 to 0.5 μm (10 to 500 nm), and the pitches thereof to 0.1 to 1 μm (100 to 1000 nm).

When the periodic fine structure of the culture substrate 1 of the present disclosure is formed by arranging the periodic lattice grooves 2b in the order of micrometers and the periodic projections 3b in the order of nanometers, it is preferable to set the groove widths of the periodic lattice grooves 2b, in the order of micrometers, to 1 to 20 μm and particularly 5 to 10 μm, the depths thereof to 0.3 to 2 μm and particularly 0.6 to 1 μm, and the pitches thereof to 1 to 100 μm and particularly 10 to 20 μm, and it is preferable to set the projection diameters of the periodic projections 3b, in the order of nanometers, to 0.1 to 1 μm (100 to 1000 nm), the heights thereof to 0.01 to 0.5 μm (10 to 500 nm), and the pitches thereof to 0.1 to 1 μm (100 to 1000 nm). Specifically, the groove widths of the periodic lattice grooves 2b in the order of micrometers are set to 6 μm, the depths thereof are set to 0.6 μm, and the pitches thereof are set to 12 μm. Furthermore, the diameters of the periodic projections 3b in the order of nanometers are set to 0.6 μm (600 nm), the heights thereof are set to 0.2 μm (200 nm), and the pitches thereof are set to 0.5 to 0.8 μm (500 to 800 nm) and particularly preferably 0.7 μm (700 nm). The density of the periodic nanoprojections is set to preferably one million to three million projections per cm² and particularly preferably a little less than two million projections per cm².

Here, the “groove width” refers to a distance between one end and the other end of the groove in a direction orthogonal to the longitudinal direction of the groove. Also in a case where the distance changes in the height direction of the groove, the “groove width” means such a distance on the surface of the culture substrate 1. The “height of the groove” means a distance between the average height of the lowermost surfaces of the grooves and the average height of the uppermost portions thereof. The “groove pitch” means an interval between a groove and its closest groove in the direction orthogonal to the longitudinal direction of the groove, and the distance of the interval corresponds to the length of a period of the recessed portion and the raised portion in the direction orthogonal to the longitudinal direction of the groove. In the case of lattice grooves, when a region surrounded by the grooves has a rectangular shape, for example, the pitch is an interval between the closest opposing grooves in the region surrounded by the grooves.

The “diameter of a projection” means a distance between one end and the other end of the projection. The diameter of a rectangular projection is a distance of one side of the projection, and the diameter of a circular projection is a distance of the diameter of the projection. Also in a case where the distance changes in the height direction of the projection, the “diameter” means such a distance on the surface of the culture substrate 1. The “height of the projection” means a distance between the average height of the lowermost surfaces of the projections and the average height of the uppermost portions thereof. The “projection pitch” means an interval between a projection and its closest projection, and the distance of the interval corresponds to the length of a period of the recessed portion and the raised portion on the surface of the culture substrate 1. It is preferable that the dimensions of the projection are calculated as the averages of measurement values of the diameters, heights, pitches, and the like of the projections existing in a predetermined region such as a region of 1 mm².

There is no limitation on the directional property of the culture substrate 1 of the present disclosure, and the periodic fine structure may be isotropic or anisotropic. Here, “isotropic” means that the arrangement of the elements of the periodic fine structure is independent of directions, and “anisotropic” means that the arrangement of the elements is dependent on directions. For example, it can be said that the hybrid periodic grooves 4a are anisotropic, and the hybrid periodic lattice grooves+periodic projections 4b are isotropic.

Although there is no limitation on the order in which the periodic microstructure 2 and the periodic nanostructure 3 are formed on the culture substrate 1 of the present disclosure, it is preferable that first, the periodic microstructure 2 is formed, and the periodic nanostructure 3 is then formed to overlap the periodic microstructure 2. Therefore, the periodic nanostructure 3 is formed on the periodic microstructure 2.

For example, the periodic grooves 3a in the order of nanometers having groove widths of 0.3 μm, depths of 0.2 μm, and pitches of 0.5 to 0.8 μm are formed to overlap the periodic grooves 2a in the order of micrometers having groove widths of 6 μm, depths of 0.9 μm, and pitches of 12 μm, thus making it possible to form the hybrid periodic grooves 4a in which the periodic grooves 2a in the order of micrometers having groove widths of 6 μm, depths of 0.6 μm, and pitches of 12 μm, and the periodic grooves 3a in the order of nanometers having groove widths of 0.3 μm (300 nm), depths of 0.2 μm (200 nm), and pitches of 0.5 to 0.8 μm (500 to 800 nm) coexist. The hybrid periodic lattice grooves+periodic projections 4b can also be formed by forming the periodic projections 3b in the order of nanometers to overlap the periodic lattice grooves 2b in the order of micrometers in the same manner.

The periodic fine structure can be formed using a known method. For example, the periodic microstructure 2 and periodic nanostructure 3 of the present disclosure can be formed using an ultra-short pulse laser with a pulse width of several femtoseconds to several picoseconds. It is preferable to use a laser processing apparatus with a wavelength in a near-infrared region of 800 nm to 1500 nm, a pulse time width of 10 ps or less, and an output of 1 W or more as the ultra-short pulse laser, and a femtosecond pulse laser is particularly preferable. FCPA pJewel D-1000 manufactured by IMRA America, Inc. can be preferably used as the femtosecond pulse laser. The periodic microstructure 2 can also be formed through mechanical processing.

The periodic microstructure 2 can be formed by non-thermal cutting using the ultra-short pulse laser, for example. When the periodic microstructure 2 is formed as periodic grooves on a titanium material, it is preferable that the wavelength is 800 to 1500 nm, the fluence is 0.1 to 1.5 J/cm², the pulse line density is 100 to 1000 pulses/mm, the scanning frequency is 1 to 50 times, and circularly polarized light or linearly polarized light is used as polarized light. It is particularly preferable that the fluence is 0.7 J/cm², the pulse line density is 300 pulses/mm, the scanning frequency is 20 times, and circularly polarized light is used as polarized light. When the periodic nanostructure 3 is formed, a periodic groove structure can be formed in a direction orthogonal to the polarization direction using linearly polarized light emitted by the ultra-short pulse laser, a granular structure can be formed using circularly polarized light, and a ridge structure can be formed using elliptically polarized light, for example. When the periodic nanostructure 3 is formed as periodic grooves on a titanium material, it is preferable that the wavelength is 800 to 1500 nm, the fluence is 0.5 to 1.5 J/cm², the pulse line density is 100 to 1000 pulses/mm, and the scanning frequency is 1 to 10 times. It is particularly preferable that the fluence is 0.8 J/cm², the pulse line density is 200 pulses/mm, the scanning is performed once, and linearly polarized light is used as polarized light. When the periodic nanostructure 3 is formed as periodic projections on a titanium material, circularly polarized light is used as the polarized light in the above-mentioned conditions.

The periodic fine structure is formed by irradiating the surface of a substrate with a laser beam such that desired shapes are drawn on the surface using the laser beam.

Scanning by the laser beam can be performed using any method such as a raster scan method, a vector scan method, and a spot scan method, and the raster scan method is preferable.

Culturing of cells using a culture substrate according to an embodiment disclosed here will be explained with reference to the attached drawings.

The culture substrate 1 of the present disclosure is a substrate for culturing stem cells, and stem cells are cultured using the surface on which the hybrid periodic structure 4 is formed as a culture surface. In order to achieve regenerative medicine, stem cells are proliferated in a state in which the undifferentiation property is maintained (which may be referred to as “undifferentiated proliferation step” hereinafter), and subsequently, the differentiation of the proliferated stem cell is induced to differentiate the stem cells into desired cells (which may be referred to as “differentiation inducing step” hereinafter). In order to regenerate tissue and viscera that can be used in transplant treatment, a vast number of stem cells are required, and it is necessary to efficiently induce the differentiation of these stem cells into desired cells. With the culture substrate 1 of the present disclosure, the proliferation and the differentiation of stem cells can be controlled appropriately.

Stem cells are undifferentiated cells having the ability to differentiate and the ability to self replicate. Here, the “ability to differentiate” means the ability to change into various types of cells that constitute tissue and viscera and have specific functions. Cells in the body have certain functions and certain shapes, and that is, the “ability to differentiate” means the ability of stem cells to change into cells having certain functions and certain shapes. From the viewpoint of the ability to differentiate, stem cells may have multipotency that allows the cells to change into all types of cells in the body, or stem cells may have the ability to change into some types of cells. Here, “undifferentiation” means a state in which cells are not differentiated into somatic cells and germ cells having specific functions and specific shapes. The “ability to self replicate” means the ability of cells to generate cells that are identical to themselves while repeating cell division.

Stem cells are cells that are not terminally differentiated, and include all cells having the ability to differentiate and the ability to self replicate. Therefore, stem cells also include cells having the ability to differentiate that are generated during a process of the terminal differentiation of the stem cells, as long as the cells have the ability to differentiate and the ability to self replicate. Examples of stem cells include induced pluripotent stem cells (abbreviated as “iPS cells” hereinafter), embryonic stem cells (abbreviated as “ES cells” hereinafter), nuclear transfer embryonic stem cells (abbreviated as “ntES cells” hereinafter), somatic stem cells, and cord blood stem cells, but there is no limitation thereto. There is a hierarchy in stem cells. iPS cells and ES cells that are higher up in the hierarchy have high ability to self replicate and can be differentiated into various cell lines, but as somatic stem cells, the lower the class of cells is, the lower the self-replication property is, and such cells at the lower class can be differentiated into some specific cell lines.

iPS cells are pluripotent stem cells that are artificially induced by introducing specific genes into somatic cells, which have originally lost the ability to differentiate. ES cells are pluripotent stem cells obtained by culturing inner cell mass in an embryo of a fertilized ovum at a blastocyst stage. ntES cells are pluripotent stem cells obtained by introducing a nucleus of a somatic cell into an ovum from which a nucleus has been removed to produce an embryo and culturing inner cell mass in the embryo in the same manner as ES cells. iPS cells can be obtained based on the methods described in Takahashi K. et al., “Induction of pluripotent stem cells from mouse embryonic and adult fibroblast cultures by defined factors.”, Cell, 126(4), 663-676 (2006); Takahashi K. et al., “Induction of pluripotent stem cells from adult human fibroblasts by defined factors.” Cell, 131(5), 861-872 (2007); Yu J. et al., “Induced Pluripotent Stem Cell Lines Derived from Human Somatic Cells”, Science, 318(5858), 1917-1920 (2007); Nakagawa M. et al., “Generation of induced pluripotent stem cells without Myc from mouse and human fibroblasts”, Nat Biotechnol., 26(1), 101-106, (2008); and the like. ES cells can be obtained based on the descriptions in M. J. Evans et al., “Establishment in culture of pluripotential cells from mouse embryos”, Nature, 292, 154-156 (1981); Thomson J A et al., “Embryonic stem cell lines derived from human blastocysts.”, Science, 282(5391), 1145-1147 (1988); Amit, M. et al., “Clonally derived human embryonic stem cell lines maintain pluripotency and proliferative potential for prolonged periods of culture.”, Dev. Biol. 227(2), 271-278 (2000); and the like. Commercially available iPS cells and ES cells or iPS cells and ES cells available from cell banks may be used.

iPS cells and ES cells are pluripotent stem cells having the ability to differentiate into a three germ layers, namely an ectoderm, a mesoderm and an endoderm, and all types of cells generated by the differentiation of the three germ layers. Pluripotent stem cells can be differentiated into cells constituting all tissue and viscera excluding a placenta and an amnion. For example, an ectoderm is differentiated into nerve cells, axons, and marrow sheaths of the nervous system; an oral epithelium, a tongue, and tooth enamel of the digestive system; skin, a cornea, a retina, an internal ear, and an external ear of the sensory system; and the like. An mesoderm is differentiated into bone, cartilage, and the like of the skeletal system; a heart, vascular endothelial cells, blood cells such as leukocytes, platelets, and erythrocytes, a spleen, bone marrow, and the like of the circulatory system; nervous microglial cells of the nervous system; a kidney and a ureter of the urinary system; an ovary, a womb, and a testis of the reproductive system; a connective tissue; and the like. An endoderm is differentiated into an esophageal epithelium, a stomach epithelium, a liver, and a pancreas of the digestive system; a thyroid gland and a thymus of the endocrine system; an auditory tube and a tympanic cavity of the sensory system; and an amygdala, a pharyngeal epithelium, a laryngeal epithelium, a tracheal epithelium, and a lung of the respiratory system; and the like.

Somatic stem cells are cells that exist in the living organism and are not terminally differentiated, and include various types such as mesenchymal stem cells (which may be abbreviated as “MSCs” hereinafter), nervous stem cells, hematopoietic stem cells, liver stem cells, vascular endothelial stem cells, and epithelial stem cells. Somatic stem cells can also be generated during a process of the terminal differentiation of iPS cells and ES cells. Unlike the pluripotent stem cells, somatic stem cells can be differentiated into only cells that constitute specific tissue and viscera.

An MSC is one type of somatic stem cell and has the ability to differentiate into a stromal cell (bone marrow), an osteoblast (osteocyte), a chondroblast (chondrocyte), a muscle cell, an adipocyte, a fibroblast (a tendon, a ligament), a vascular endothelial cell, and the like of mesodermal origin. Furthermore, it is reported that an MSC also has the ability to differentiate into a nerve cell of ectodermal origin, and a hepatocyte and a pancreatic cell of endodermal origin, regardless of the difference in germ layer.

MSCs can be obtained from various tissue such as bone marrow, adipose tissue, and muscle. Preferably, bone marrow mesenchymal stem cells can be obtained from bone marrow. Bone marrow mesenchymal stem cells are included in bone marrow stromal cells and can be obtained by seeding bone marrow fluid collected from a bone marrow puncture on a petri dish and proliferating fibroblastoid cells, which sediment on the bottom of the petri dish and proliferate, by passage culture, for example. Also, MSCs can be obtained by inducing the differentiation of pluripotent stem cells such as iPS cells and ES cells. For example, it is reported that MSCs can be obtained by culturing ES cells in the presence of retinoic acid and selecting SOX1-positive cells and that MSCs can be obtained by culturing ES cells and selecting PDGFRα-positive and FLK1-negative cells that have a shape like stromal cells and do not express Mesp2 (see JP 2005-304443A and WO 2004/106502). Commercially available MSCs and MSCs available from cell banks may be used.

There is no limitation on the origin of cells to be cultured using the culture substrate 1 of the present disclosure. Therefore, cells derived from mammals such as humans, monkeys, mice, rats, hamsters, rabbits, cattle, horses, pigs, dogs, cats, goats, and sheep, birds, and reptiles may be cultured. Preferably, the culture substrate 1 can be used for culturing of cells derived from mammals.

In the undifferentiated proliferation step, stem cells are cultured on the surface of the culture substrate 1 of the present disclosure on which the hybrid structure 4 is formed. In the undifferentiated proliferation step in which the differentiation is not induced, using the culture substrate 1 of the present disclosure to culture stem cells makes it possible to suppress the generation of differentiated cells generated by spontaneous differentiation or the like and proliferate the stem cells in a state in which the undifferentiation property is maintained. That is, stem cells are not differentiated into any type of cell on the culture substrate 1 of the present disclosure unless the differentiation is induced, and their karyotypes are not abnormal. On the other hand, stem cells can sufficiently exhibit the ability to self replicate to generate cells having the same characteristics as those of themselves. This makes it possible to efficiently proliferate stem cells in a state in which the undifferentiation property is maintained and to stably provide a high-quality stem cell population. Accordingly, it is possible to provide stem cells in an amount sufficient enough to provide differentiated cells in an amount sufficient enough to use the differentiated cells in regenerative medicine, a drug development screening, and the like.

There is no particular limitation on the culturing of stem cells in the undifferentiated proliferation step as long as the stem cells are maintained. Therefore, stem cells can be cultured based on methods known in the art. That is, cells can be seeded in a liquid culture medium for a primary culture and cultured in appropriate conditions. An exchange of the liquid culture medium and passage can also be performed in the same manner as in the known methods.

Specifically, there is no limitation on a culture medium, culture conditions, and the like in the culturing of iPS cells and ES cells as long as iPS cells and ES cells can be maintained, and iPS cells and ES cells can be cultured based on a known culture medium and known culture conditions. A known culture medium used for ordinary culturing of iPS cells and ES cells can be used as the culture medium. A culture medium obtained by adding a cell growth factor such as a basic fibroblast growth factor (bFGF) to a serum-free culture medium can be used, for example, and a culture medium can be selected as appropriate depending on the types of cells to be cultured. Furthermore, a commercially available culture medium for the culture of iPS cells and ES cells, such as StemPro (registered trademark) hESC (Life technologies) and ReproFF2 (Reprocell), can be used.

There is no particular limitation on a culture medium, culture conditions, and the like in the culture of MSCs as long as the MSCs are maintained, and MSCs can be cultured based on a known culture medium and known culture conditions. A known culture medium used for ordinary culturing of MSCs can be used as the culture medium. Examples of the culture medium include an MEM culture medium and a DMEM culture medium, and a culture medium can be selected as appropriate depending on the types of cells to be cultured. Furthermore, an MSC growth medium and a kit that are commercially available, such as MSCGM™ BulletKit™ (Lonza, Catalog No. PT-3001), can be used.

Culture conditions can also be selected as appropriate depending on the types of cells to be cultured. For example, cells can be seeded at the initial seeding density of 5000 to 6000 cells/cm² and cultured in an incubator in which the temperature is set to 37° C. and the CO₂ concentration is set to 5%.

It is possible to confirm whether or not stem cells maintain the ability to differentiate by the observation of the cell morphology, the confirmation of the ability to differentiate, the confirmation of an undifferentiation marker, and the like. The undifferentiation marker is a molecule that is expressed specifically in undifferentiated stem cells and plays a very important role in exhibiting the ability to differentiate, and the expression or the expression level of this molecule can be detected using a known method. For example, real-time RT-PCR or the like can be used to detect the expression of the marker gene, and an immunostaining method using a polyclonal antibody or a monoclonal antibody that is specific to the marker, an enzyme activity measurement method, or the like can be used to detect the expression of a protein marker. An immunofluorescence staining method is preferably used as the immunostaining method using an antibody. Here, the “immunofluorescence staining method” is a method for introducing an antibody labeled with a fluorescent dye into a sample to stain the sample by using an antigen-antibody reaction, and using this method makes it possible to stain the sample with high specificity against an antigen substance (undifferentiation marker).

Examples of the undifferentiation marker for pluripotent stem cells such as iPS cells and ES cells include Nanog, SRY (sex determining region Y)-box 2 (SOX2), SSEA-1, SSEA-3, SSEA-4, TRA-1-60, TRA-1-81, and OCT3/4. Examples of the undifferentiation marker for MSCs include CD29, CD44, CD71, CD73 (SH3/4), CD90 (Thy-1), CD105 (SH2), CD106, CD166, and Stro-1. A negative marker can also be used, and examples of the negative marker for MSCs include CD11b, CD14, CD19, CD31, CD18, CD34, CD45, CD56, CD79a, and HLA-DR.

) In the differentiation inducing step, the differentiation of stem cells is induced on the surface of the culture substrate 1 of the present disclosure on which the hybrid periodic structure 4 is formed. In the differentiation inducing step, using the culture substrate 1 of the present disclosure to induce the differentiation makes it possible to efficiently differentiate stem cells into desired cells on the culture substrate 1 of the present disclosure while suppressing the generation of stem cells that maintain the undifferentiation property.

Any method known in the art can be used as the method for inducing the differentiation as long as stem cells can be differentiated into desired cells. For example, the differentiation can be induced by culturing cells on the culture substrate 1 of the present disclosure using a differentiation inducing culture medium containing a differentiation inducing factor, and desired differentiated cells can be obtained by exchanging the culture medium to the differentiation inducing culture medium after the above-mentioned undifferentiated proliferation step using the culture substrate 1 of the present disclosure. Also, the culture substrate 1 of the present disclosure can be used to perform only one of the undifferentiated proliferation step and the differentiation inducing step.

The differentiation inducing factor can be selected as appropriate depending on the types of cells to be differentiated, the differentiation classes of stem cells to be differentiated, and the like. There is also no limitation on the induction conditions including the period of time of contact with the differentiation inducing factor, and the like as long as the differentiation into a desired type of cell can be induced. Furthermore, a differentiation inducing reagent and a kit that are commercially available can be used.

For example, pluripotent stem cells such as iPS cells and ES cells can be brought into contact with differentiation inducing factors of specific concentrations at specific times in a specific order to be differentiated into cells belonging to a specific cell line via a germ layer. For example, the addition of activin and a basic fibroblast growth factor (bFGF) to pluripotent stem cells enables the differentiation into a mesendoderm and an endoderm, and the addition of bone morphogenetic protein (BMP) enables the differentiation into a mesoderm.

Insulin, dexamethasone, 3-isobutyl-1-methylxanthine (IBMX), indomethacin, 3,3,5-triodothyronine (T3), and the like can be used to induce the differentiation of MSCs into adipocytes. Dexamethasone, L-glutamine, ascorbic acid, I-glycerophosphate, and the like can be used to differentiate MSCs into osteocytes. Dexamethasone, ascorbic acid, ITS (insulin, transferrin, selenium), and the like can be used to differentiate MSCs into chondrocytes. β-mercaptoethanol, dimethyl sulfoxide (DMSO), forskolin, bFGF, and the like can be used to differentiate MSCs into nerve cells. 5-Azacytidine and the like can be used to differentiate MSCs into skeletal muscle cells. ITS, dexamethasone, a hepatocyte growth factor (HGF), oncostatin, and the like can be used to differentiate MSCs into hepatocytes. Dickkoph-1 (Dkk1), insulin-like growth factor binding protein 4 (IGFBP-4), and the like can be used to differentiate MSCs into cardiac muscle cells.

Specifically, when the differentiation of MSCs into adipocytes is induced, the Induction of the differentiation can be started at the time when the confluence of MSCs reaches preferably 80 to 90%. hMSC-BulletKit™-adipogenic (Lonza, Catalog No. PT-3004) can be used as a culture medium for the induction of differentiation into adipocytes to induce the differentiation into adipocytes in accordance with the instructions of the manufacturer. This kit includes a basal medium, L-glutamine, a mesenchymal cell growth supplement (MCGS), dexamethasone, indomethacin, 3-isobutyl-1-methylxanthine (IBMX), and GA-1000 (gentamicin, amphotericin B). It is preferable that the initial cell seeding density is 2.1×10⁴ cells/cm².

When MSCs are to be differentiated into chondrocytes, the induction of the differentiation can be started at the time when the confluence of MSCs reaches preferably 100%. hMSC-BulletKit™-chondrogenic (Lonza, Catalog No. PT-3003) can be used as a culture medium for the induction of differentiation into chondrocytes to induce the differentiation into chondrocytes in accordance with the instructions of the manufacturer. This kit includes a basal medium, L-glutamine, dexamethasone, ascorbic acid, ITS+supplement, sodium pyruvate, proline, and GA-1000 (gentamicin, amphotericin 8). It is preferable that the initial cell seeding density is 5×10⁵ cells/cm².

When MSCs are to be differentiated into osteocytes, the induction of the differentiation can be started at the time when the confluence of MSCs reaches preferably 100%. hMSC-BulletKit™-osteogenic (Lonza, Catalog No. PT-3002) can be used as a culture medium for the induction of differentiation into osteocytes to induce the differentiation into osteocytes in accordance with the instructions of the manufacturer.

This kit includes a basal medium, L-glutamine, dexamethasone, ascorbic acid, ITS+supplement, sodium pyruvate, proline, a mesenchymal cell growth supplement (MCGS), β-glycerophosphate, and penicillin/streptomycin. It is preferable that the initial cell seeding density is 3.1×10⁵ cells/cm².

When MSCs are to be differentiated into nerve cells, the induction of the differentiation can be started at the time when the confluence of MSCs reaches preferably 80 to 90%. Mesenchymal Stem Cell Neurogenic Differentiation Medium (PromoCell, Catalog No. C-28015) can be used as a culture medium for the induction of differentiation into nerve cells to induce the differentiation into nerve cells in accordance with the Instructions of the manufacturer. This kit includes a basal medium and Supplement Mix (PromoCell. Catalog No. C-39815). It is preferable that the initial cell seeding density is 5000 cells/cm².

It is possible to confirm whether or not stem cells have differentiated into a desired type of cell by observing the cell morphology and confirming the expression of a differentiation marker for confirmation of differentiation that is specific to desired differentiated cells. A method known in the art can be used to confirm the expression of the differentiation marker. For example, real-time RT-PCR or the like can be used to detect the expression of the marker gene, and an immunostaining method using a polyclonal antibody or a monoclonal antibody that is specific to the marker, an enzyme activity measurement method, or the like can be used to detect the expression of a protein marker. An immunofluorescence staining method is preferably used as the immunostaining method using an antibody. Here, the “immunofluorescence staining method” is a method for introducing an antibody labeled with a fluorescent dye into a sample to stain the sample by using an antigen-antibody reaction, and using this method makes it possible to stain the sample with high specificity against an antigen substance (differentiation marker).

Specifically, a peroxisome proliferator-activated receptor γ (PPARγ (also referred to as NR1C3 or PPARG)). CCAAT/enhancer binding protein p (C/EBPP), fatty acid binding protein (FABP (also referred to as aP2)), lipoprotein lipase (LPL), and the like can be used to confirm the differentiation into adipocytes. Sex determining region Y-type high mobility group box protein 9 (SOX9), aggrecan, and the like can be used to confirm the differentiation into chondrocytes. Secretory phosphorylated protein I (SPP1 (also referred to as osteopontin (OPN))), bone sialoprotein (BSP), osteocalcin (OCN), alkaline phosphatase (ALP), calcification ability, and the like can be used to confirm the differentiation into osteocytes. Microtubule associated protein 2 (MAP2), nestin, class III βtubulin (β_{III}-tubulin), and the like can be used to confirm the differentiation into nerve cells.

Here, PPARγ is a protein belonging to a nuclear receptor superfamily, functions as a transcription factor, is mainly distributed in adipose tissue, and relates to the induction of the differentiation of preadipocytes into adipocytes. SOX9 plays an essential role in the aggregation of undifferentiated mesenchymal cells and subsequent processes of the differentiation into chondrocytes. On the other hand, SOX5 and SOX6 are induced by SOX9, and these three proteins induce the transcription of a cartilage specific gene such as type-ii collagen and determine the differentiation into chondrocytes in a coordinated manner. SPP1 relates to the adhesion of osteoclasts to a calcified bone matrix, and the expression level of SPP1 increases at an early stage and an intermediate stage of the differentiation of osteoblasts. MAP2 is a microtubule binding protein that exists in neurons of vertebrates, in a large amount. The expression of MAP2 starts when neural precursor cells are differentiated, and in mature neurons, substantially no MAP2 exists in axons, whereas MAP2 is localized substantially specifically in dendrites and cell bodies.

The hybrid structure 4, which simultaneously includes the periodic microstructure 2 and the periodic nanostructure 3, is formed on the surface of the culture substrate 1 of the present disclosure. When the two periodic fine structures are formed individually, biocompatibility and biological cell affinity are slightly improved, whereas when the two periodic fine structures coexist, the biocompatibility and biological cell affinity are particularly improved. Moreover, with the culture substrate 1 of the present disclosure, stem cells can be efficiently proliferated in a state in which the undifferentiation property is maintained, thus making it possible to stably provide a high-quality stem cell population. Moreover, the culture substrate 1 of the present disclosure contributes to the induction of the differentiation of stem cells as well as promoting the induction of the differentiation of stem cells induced by a differentiation inducing factor, and the periodic fine structure formed on the surface of the culture substrate 1 closely relates to the induction direction of the differentiation of stem cells, thus making it possible to induce the differentiation of stem cells into a desired type of cell.

The periodic fine structure of the culture substrate 1 of the present disclosure can be formed in a simple manner with scanning using an ultra-short pulse laser, for example, which has an advantage in that there is little restriction on the production because processing can be performed in atmospheric air due to little thermal influence, for example. Accordingly, the culture substrate 1 of the present disclosure can be produced in a simple manner at low cost. In addition, the culture substrate 1 of the present disclosure can be produced by forming the periodic fine structure on a titanium material having high biocompatibility and biological cell affinity, and therefore, further improvement in biocompatibility and biological cell affinity can be expected.

The culture substrate 1 of the present disclosure having such characteristics can be preferably used in technical fields using stem cells. For example, cells, tissue, and organs generated by culturing stem cells on the culture substrate 1 of the present disclosure are applied to a pharmacological test and a drug development screening in which the efficacy, pharmacokinetics, safety, and the like of a development candidate drug are evaluated; the clarification of a development mechanism, a differentiation mechanism, and a disease mechanism; and regenerative medicine and cell therapy in which the functions of impaired viscera and organs are regenerated, and thus the contribution to drug development, life science, and medical treatment is expected. In the field of regenerative medicine, for example, the differentiation into chondrocytes can be applied to the treatment of osteoarthritis and the like, the differentiation into tendon cells can be applied to the treatment of ligament rupture, the differentiation into osteocytes can be applied to the treatment of intractable bone fractures and to implants such as artificial joints and artificial tooth roots, adipocytes can be applied to the regeneration of breasts, and cardiac muscle cells can be applied to the treatment of angina pectoris, cardiac infarction, and the like, thus making it possible to contribute to the development of tailored treatment.

It was confirmed that the periodic fine structure formed on the culture substrate 1 of the present disclosure contributes to the induction of differentiation, and thus the differentiation of a plurality of cells can be simultaneously induced by appropriately controlling the pattern of the periodic fine structure formed on the culture substrate 1. Accordingly, a plurality of types of cells are systematically arranged and constructed, thus making it possible to apply the culture substrate 1 of the present disclosure to the regeneration of highly functional viscera and organs.

### EXAMPLES

Hereinafter, the present disclosure will be described in detail by way of examples, but the present disclosure is not limited to the following examples. It should be noted that Examples 1 to 10 disclose examples in which an anisotropic culture substrate 1 having a structure with hybrid periodic grooves 4a obtained by forming the periodic grooves 2a in the order of micrometers as the periodic microstructure 2 and forming the periodic grooves 3a in the order of nanometers as the periodic nanostructure 3 to overlap the periodic grooves 2a was produced and used to induce the differentiation of mesenchymal stem cells into osteocytes and chondrocytes (I). Examples 11 and 12 disclose examples in which an isotropic culture substrate 1 having a structure with the hybrid periodic lattice grooves+periodic projections 4b obtained by forming the periodic lattice grooves 2b in the order of micrometers as the periodic microstructure 2 and forming the periodic projections 3b in the order of nanometers as the periodic nanostructure to overlap the periodic lattice grooves 2b was produced and used to induce the differentiation of mesenchymal stem cells into osteocytes, chondrocytes, nerve cells, and adipocytes (II).

I. Examination Using Culture Substrate 1 on which Hybrid Periodic Grooves 4a are Formed

### Example 1

### Examination of Production of Culture Substrate 1 (Preliminary Examination)

1. Summary

In this example, the production of a culture substrate in which a periodic fine structure was formed on the surface was examined in order to construct a culture substrate with which biocompatibility is improved.

Titanium rarely causes an immune response when embedded in a living organism, and thus is widely used for implant materials such as artificial joints and artificial dental roots. However, the field of compatibility of titanium materials with soft tissue has not been examined yet, and it has been reported that implants have been extracted due to incompatibility, for example. Therefore, in this example, the production of a culture substrate in which a periodic fine structure was formed on the surface through laser processing was examined in order to explore the possibility of improving the biocompatibility by forming the periodic fine structure. A preliminary examination was performed in this example.

Specifically, it was examined that a structure in which grooves in the order of micrometers having a width of 5 to 10 μm, a depth of 1 μm, and a pitch of 10 to 20 μm were periodically arranged (which may be also referred to as “periodic microgrooves” hereinafter), a structure in which grooves formed to have a certain width (in the course of nature), a certain depth (in the course of nature), and a pitch (<1 μm) in the order of nanometers were periodically arranged (which may be also referred to as “periodic nanogrooves” hereinafter), and a structure in which the above-mentioned periodic microgrooves and periodic nanogrooves were combined (which may be referred to as “hybrid periodic grooves” hereinafter) were respectively formed on the surfaces of titanium plates.

2. Materials and Methods

2-1. Substrate

A mirror polished titanium plate of JIS class 2 (a plate of φ14 mm×1 mm or <φ8×1 mm in which one side is polished) was used as a substrate for the production of a culture substrate. A titanium material that was cut out from a grade-2 pure titanium round bar and polished into a mirror surface was purchased from TDC Corporation (24-15 Chojamae, lidol, Rifu-cho, Miyagl 981-0113, JAPAN).

2-2. Laser

A femto-second laser was used as a laser for forming a periodic fine structure on the surface of a substrate. FCPA pJewel D-1000 (referred to as “D-1000” hereinafter) or FCPA pJewel Test Laser (referred to as “Test Laser” hereinafter) manufactured by IMRA America, Inc. was used as the femto-second laser. Details of the lasers are summarized in Table 1 below.

TABLE 1

| Apparatus | D-1000 | Test Laser |
|---|---|---|
| Ossilation | 1040 nm | 520 nm |
| wavelength | (fundamental wave) | (second high frequency: SHG) |
| Repetition | 100 KHz | 100 kHz |
| frequency | | |
| Output | 1.2 W | 0.34 W |
| | (fundamental wave) | (second high frequency: SHG) |
| Pulse width | 400 fs | 1680 fs |
| M² | 1.1 | unknown |

3. Results

As a result of inputting a full-power single pulse from D-1000 to a titanium plate, a processing trace had a substantially circular shape having a diameter of about 2 μm, and burrs appeared to scatter around the processing trace. When the same irradiation conditions were used and the moving speed of the stage was reduced such that pulses overlapped each other, a processing trace had such a shape that the melt scattered due to its surface being struck. It was thought from these results that non-thermal processing was not achieved in the case of the input of 1.2 W, and titanium existed in a melt-like state for at least about 10 μs. A similar tendency was confirmed when the periodic nanogroove structure was formed, and therefore, a threshold value at which non-thermal processing on titanium can be achieved was examined.

Processing states were compared using the fluence of Test Laser (SHG) as a parameter (1.00 J/cm², 0.64 J/cm², 0.32 J/cm², and 0.16 J/cm²). When single scan processing using a galvano scanner was performed in order to separate individual processing traces, it was confirmed that such a structure that began to melt was formed in the cases of fluences of the upper limit to 0.32 J/cm². On the other hand, in the case of the fluence of 0.16 J/cm², the processing trace was in a state in which the coating on the surface partially came off.

An attempt was made to perform repetitive sweeps at the same position in order to confirm whether or not non-thermal processing was achieved in the case of Test Laser (SHG) with 0.16 J/cm². As a result, it was confirmed that the periodic nanogroove structure was formed in V-shaped grooves and wall surfaces, and no shear drop that is observed when a structure is melted, and the like was formed. These are the characteristics of general non-thermal processing. It was thought from these results that a threshold value at which non-thermal processing could be achieved existed but was saturated with an extremely low fluence, resulting in a transition to a thermal processing mode.

In view of these results, it was thought that it was necessary to perform sweeping processing with extremely small input and determine the number of pieces of processing with which a predetermined depth can be obtained in order to perform processing for forming the periodic microgroove structure. Moreover, the threshold value of Test laser (SHG) was thought to be about 0.20 J/cm².

The conditions for the formation of the periodic nanogrooves were examined using D-1000 (fundamental wave) and Test Laser (SHG). Although it is generally assumed that a periodic nanogroove structure is formed to have a period of 70 to 80% of a wavelength, the periodic nanogroove structure having a period of about 150 nm, which is about ⅓ of the wavelength, was formed with Test Laser (SHG). When the fluence was reduced to such an extent that the thermal influence could be eliminated, a region capable of being processed by one scan had a narrow width of about 1 μm, and thus a portion in which the structure was formed and a portion in which no structure was formed coexisted in the input range, for example. It was thus revealed that there were a large number of problems in using such a fluence as a stability condition.

As a result of examining a fundamental wave threshold value in the same manner as that of SHG, a threshold value of 1.50 J/cm² was obtained. It is assumed that the reason why the value differed from that of SHG is that an energy amount that is converted into heat is small in the case of the fundamental wave due to the difference in linear absorption to titanium. In the formation using D-1000 (fundamental wave), a periodic nanogroove structure having a period of 70 to 80% of an input wavelength was formed, and the range in which the structure was formed is wide and stable.

It was determined from the above-described results of the preliminary examination that it is difficult to perform stable processing on a titanium material using a laser with a wavelength of 520 nm at present, and therefore, a fundamental wave laser with a wavelength of 1040 nm was used in the following examples.

### Example 2

### Examination of Production of Culture Substrate (Determination of Laser Processing Conditions)

1. Summary

In this example, the examination of a threshold value of the processing and the adjustment of the shape were performed based on the results of the preliminary examination in Example 1. and thus laser processing conditions were determined.

2. Determination of Processing Conditions

Table 2 below shows the parameters after the determination of the processing conditions. The conditions for a fundamental wave laser were determined based on the above-mentioned results of the preliminary examination in Example 1.

TABLE 2

| | | | | (3) Hybrid |
|---|---|---|---|---|
| | | (1) Periodic | (2) Periodic | periodic |
| | | microgrooves | nanogrooves | grooves |
| Used laser | | D-1000 | | |
| Power | | 0.025 W | 0.25 W | (1) + (2) |
| | | (0.25 μJ/pulse) | (2.5 μJ/pulse) | |
| Repetition frequency | | 100 kHz | | |
| Fluence | | 0.7 J/cm² | 0.8 J/cm² | (1) + (2) |
| Scanning conditions | | 300 mm/ | 500 mm/ | (1) + (2) |
| | | second, | second, | |
| | | 20 sweeps, | pitch of 10 μm | |
| | | pitch of 12 μm | | |
| Optical element | | λ/4, DOE | λ/2, DOE | (1) + (2) |
| | | (top hat) | (top hat) | |
| Processing | Groove width | 6 μm | 0.3 μm | (1) + (2) |
| result | Groove depth | 0.9 μm | 0.2 μm | 0.6 μm |
| | Groove pitch | 12 μm | 0.5 to 0.8 μm | (1) + (2) |

3. Results

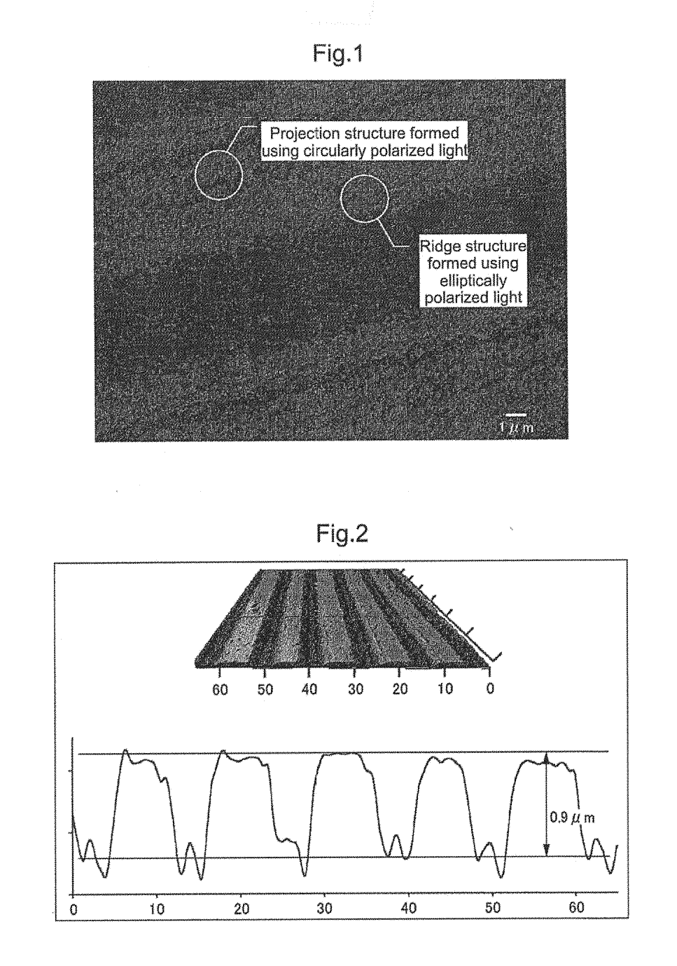

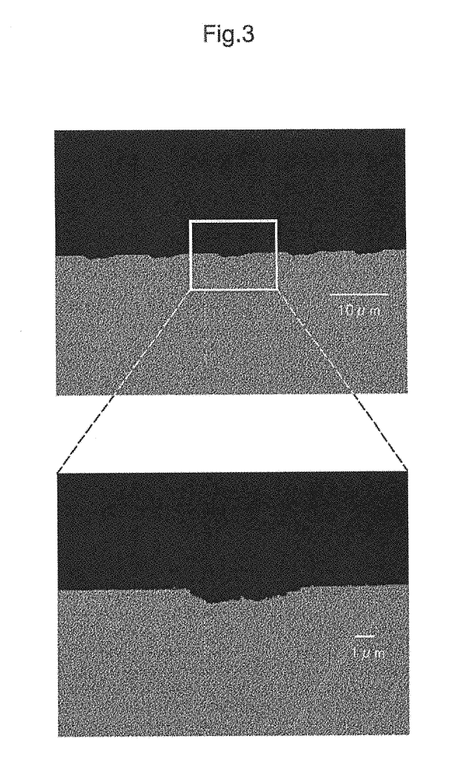

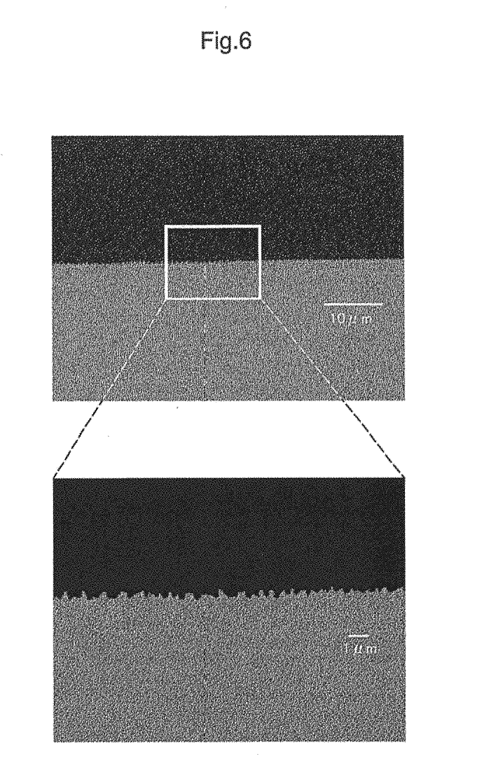

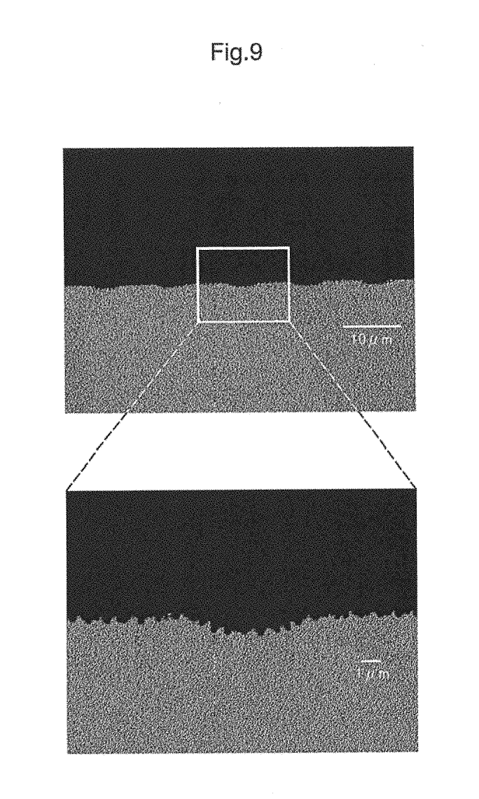

FIGS. 1 to 9 show scanning electron microscope (SEM) images of the processed surface, the results of the depth observation under a laser microscope, and SEM images of the cross sections, in the above-mentioned processing conditions.

FIGS. 1 to 3 respectively show the SEM image of the processed surface, the observation result of the depth, and the SEM images of the cross sections, of the periodic microgrooves. The reason why a λ/4 wavelength plate was used as an optical element in the conditions for the production of periodic microgrooves is to prevent the generation of anisotropy due to the formation of the periodic nanostructure on the processed surface, and a periodic ridge nanostructure formed using linearly polarized light changes into such a periodic projecting nanostructure that is similar to the bottom portion of the processed surface shown in FIG. 1. However, during the input to the inclined surface, circularly polarized light ovalizes, and thus the generation of anisotropy cannot be prevented. Therefore, a ridge structure is generated on the processed edge portion. The reason why a diffraction optical element (DOE) was used is that it is difficult to increase the processing width while suppressing the peak intensity of a Gaussian beam to a level smaller than or equal to a non-thermal processing threshold, and the beam intensity was averaged all over the surface. As a result of the processing, a structure that met the required width, depth, and pitch could be provided. However, since the DOE was used, the shape of the top hat changes at a position separated from the focus, and therefore, care needs to be taken.

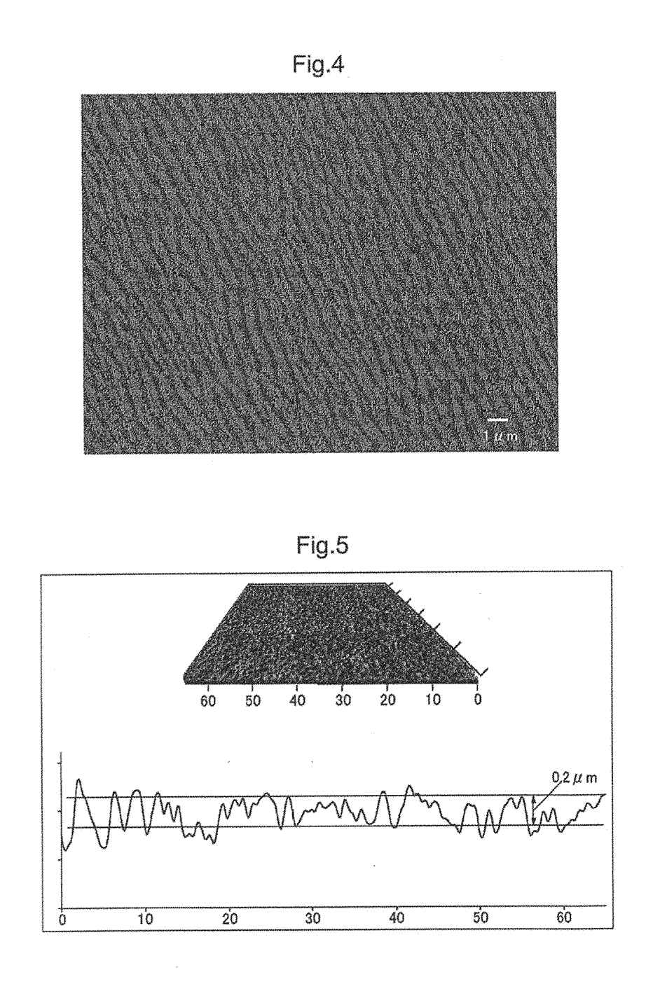

FIGS. 4 to 6 respectively show the SEM image of the processed surface, the observation result of the depth, and the SEM images of the cross sections, of the periodic nanogrooves. The reason why a λ/2 wavelength plate was used as an optical element in the conditions for the production of periodic nanogrooves is that it is necessary to align the orientation of the ridges of the periodic nanogrooves with the orientation of the periodic microgrooves when producing the hybrid periodic grooves. As a result of the processing, a structure having an average groove pitch of about 0.7 μm could be provided.

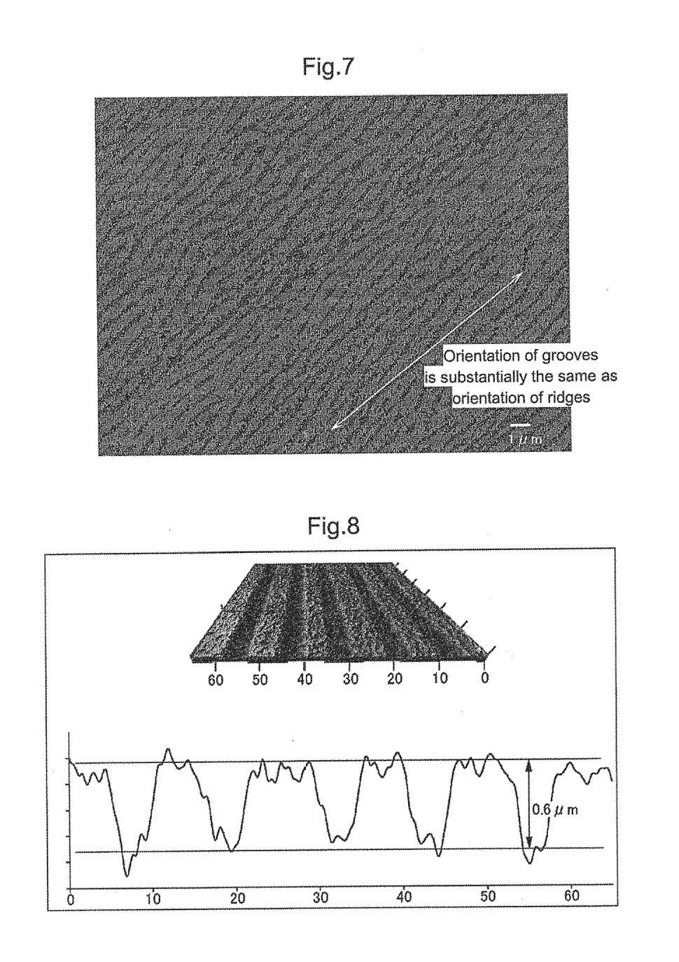

FIGS. 7 to 9 respectively show the SEM image of the processed surface, the observation result of the depth, and the SEM images of the cross sections, of the hybrid periodic grooves. The hybrid periodic grooves were formed in accordance with the procedure in which, first, the periodic microgrooves were formed and the periodic nanogrooves were then formed to overlap the periodic microgrooves. As a result of the processing, a structure in which the characteristics of the periodic microgrooves and the periodic nanogrooves coexist could be obtained, and the periodic microgrooves extended in substantially the same direction as the periodic nanogrooves. Only the depth of the grooves was unlike that of the periodic microgrooves, that is, shallower than that of the periodic microgrooves, and it is thought that this is because the structure was loosened during the process of forming the periodic nanogrooves.

### Example 3

### Biological Cell Affinity Testing (Calculation of Cell Density)

1. Summary

In this example, biological cell affinity testing was performed on the culture substrate in which the periodic fine structure was formed on the surface based on the processing conditions determined in Example 2 above. The biological cell affinity was evaluated in view of a cell density in this example.

2. Materials and Methods

2-1. Culture Substrate

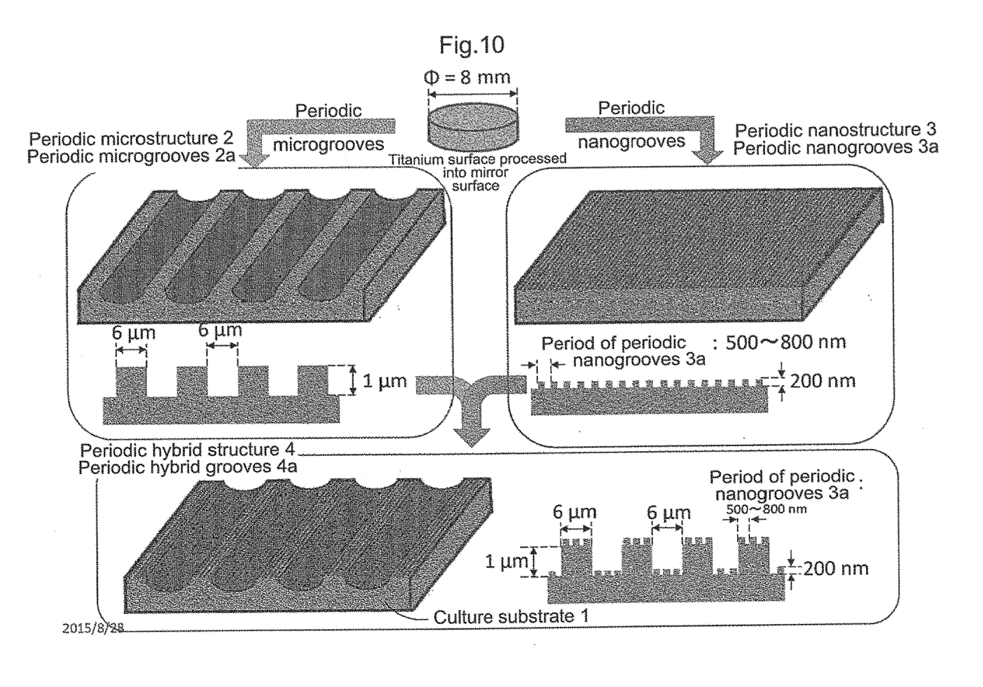

A medical titanium plate (φ8 mm×1 mm) in which one side was polished into a mirror surface was used as a substrate in the same manner as in Examples 1 and 2, and culture substrates 1 in which the periodic microgrooves 2a, the periodic nanogrooves 3a, and the hybrid periodic grooves 4a were respectively formed on the substrate surfaces using a femto-second laser D-1000 based on the conditions determined in Example 2 above were produced. FIG. 10 shows schematic views of the culture substrates 1 produced in this example.

2-1-1. Culture Substrate in which Periodic Microgrooves were Formed on the Surface

The periodic microgrooves were formed by irradiating and scanning the surface of the above-mentioned medical titanium plate with a femto-second laser beam (fluence: 1.4 J/cm², scanning speed: 300 mm/second, scanning frequency: 14 times, polarized light: circularly polarized light). As a result, periodic microgrooves having a width of 6 μm, a depth of 1 μm, and a pitch of 12 μm were formed. Here, the “pitch” means the length of a period of the recessed portion and the raised portion in a direction orthogonal to the longitudinal direction of the grooves, and since the groove width was 6 μm, unprocessed portions had a width of 6 μm.

2-1-2. Culture Substrate in which Periodic Nanogrooves were Formed on the Surface

The periodic nanogrooves were formed by Irradiating and scanning the surface of the above-mentioned medical titanium plate with a femto-second laser beam (fluence: 3.2 J/cm², scanning speed: 500 mm/second, scanning frequency: once, polarized light linearly polarized light). As a result, periodic nanogrooves having a depth of 0.2 μm and a pitch of 0.5 to 0.8 μm were formed.

2-1-3. Culture Substrate 1 in which Hybrid Periodic Grooves 4a were Formed on Surface

The hybrid periodic grooves 4a in which the periodic microgrooves 2a and the periodic nanogrooves 3a coexisted were formed by irradiating and scanning the surface of the above-mentioned medical titanium plate with a femto-second laser beam. First, the periodic microgrooves 2a were formed in accordance with the procedure described in 2-1-1. above, except that the scanning frequency was set to 20 times, and then, the periodic nanogrooves 3a were formed to overlap the periodic microgrooves 2a in accordance with the procedure described in 2-1-2. above. In the case of periodic nanogrooves, the orientation of the polarized light was orthogonal to the scanning direction. As a result, a culture substrate 1 having the hybrid periodic grooves 4a obtained by forming the periodic nanogrooves 3a having a depth of 0.2 μm and a pitch of 0.5 to 0.8 μm on the periodic microgrooves 2a having a groove width of 6 μm. a depth of 1 μm, and a pitch of 12 μm was produced.

2-1-4. Control

A mirror polished medical titanium plate was used as it was without performing laser processing (which may be referred to as “mirror surface” hereinafter).

2-2. Cells

Human MSCs (hMSCs, mesenchymal stem cells, Lonza, Catalog No. PT-2501) were used.

3. Testing Method

Each of the above-mentioned culture substrates was immersed in 70% ethanol for 20 minutes to be sterilized, and then cleaned three times using distilled water. After being cleaned, the culture substrate was left to stand on the bottom surface of a well of a 12-well cell culture plate, a culture medium was poured into the well, and thus the culture substrate was immersed in the culture medium. MSCs were seeded on each of the culture substrates immersed in the culture medium and cultured for six hours.

At this time, the initial cell seeding density was 5000 cells/cm². MSCGM™ BulletKit™ (Lonza, Catalog No. PT-3001) was used as the culture medium. This kit includes a basal medium for mesenchymal stem cells into which a SingleQuots™ proliferation supplement is added (Mesenchymal stem cell basal medium plus Single Quots™ of growth supplements). including a basal medium (Lonza, Catalog No. PT-3238), a mesenchymal cell growth supplement (MCGS, Lonza, Catalog No. PT-4105), L-glutamine, and GA-1000 (gentamicin, amphotericin B).

After being cultured, cells were peeled off from each of the culture substrates and collected, and the number of cells was measured using Cell Counting Kit-8 (CCK-8). Specifically, after performing a color reaction using a CCK-8 solution, the absorbance at 450 nm (reference wavelength: 630 nm) was measured using a microplate reader, and then the number of cells adhering to each of the culture substrates was calculated.

4. Results

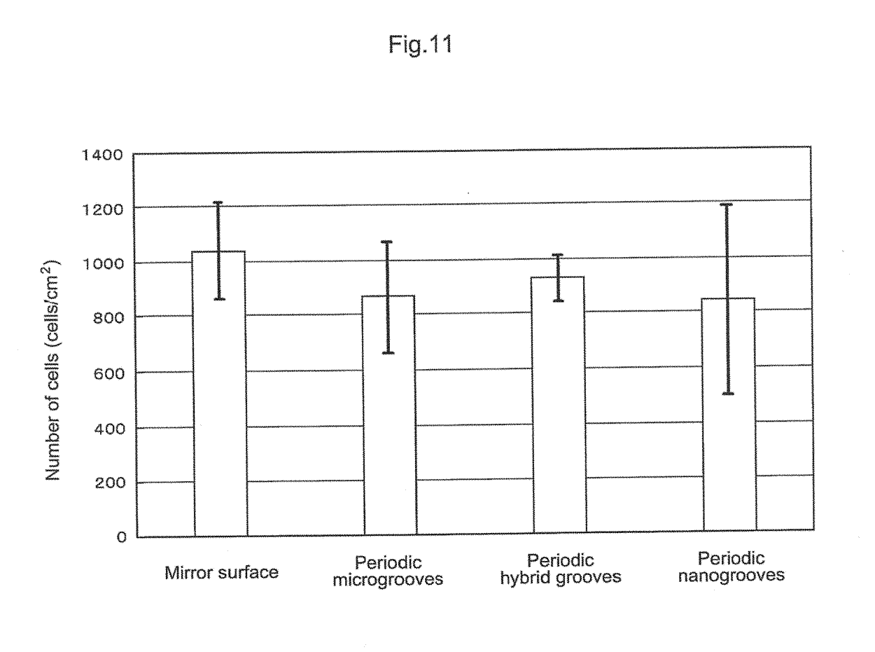

The graph in FIG. 11 shows the results. In all of the cases including the case of the control culture substrate (mirror surface), the case of the culture substrate on which the periodic microgrooves were formed, the case of the culture substrate on which the periodic nanogrooves were formed, and the case of the culture substrate 1 on which the hybrid periodic grooves 4a in which the periodic microgrooves 2a and the periodic nanogrooves 3a coexisted were formed, differences in the density of the cells adhering to the culture substrate were not observed.

### Example 4

### Biological Cell Affinity Testing (Observation of Cell Morphology)

1. Summary

In this example, biological cell affinity testing was performed on the culture substrates in which the periodic fine structure was formed on the surface, the culture substrates being produced in Example 3 based on the processing conditions determined in Example 2 above. The biological cell affinity was evaluated in view of a cell morphology in this example.

2. Testing Method

MSCs were seeded on the culture substrates and cultured for six hours in the same manner as in Example 3 above. After the culturing, the culture substrates were removed from the culture medium, and immobilized by formalin treatment.

Subsequently, immunofluorescence staining was performed, and then the cell morphology were observed under a fluorescent microscope.

The immunofluorescence staining was performed in accordance with the following procedure.

a. Staining of Cytoskeleton

F-actin was stained using rhodamine-labeled phalloidin (phalloidin-rhodamine) to observe cytoskeletons. Here, actins form a spiral polymer constituting an actin filament, which is one type of microfilament. The actin filament is the thinnest among the three types of cytoskeletons that form a three-dimensional fibrous structure, that is, an actin filament, a microtubule, and an intermediate filament, and determines the cell morphology, thus making it possible to check the cytoskeleton structure by staining actins.

b. Staining of Cell Nucleus

Cell nuclei were stained using 4′,6-diamidino-2-phenylindole dihydrochloride (DAPI) and observed.

c. Staining of Desmosome

Vinculin was stained to observe desmosomes. The desmosome is one type of structure via which a cell adheres to another cell and a substrate, and is classified as an adhesion complex within the framework of a cell junction.

d. Merge

The staining results in a to c above were merged.

3. Results

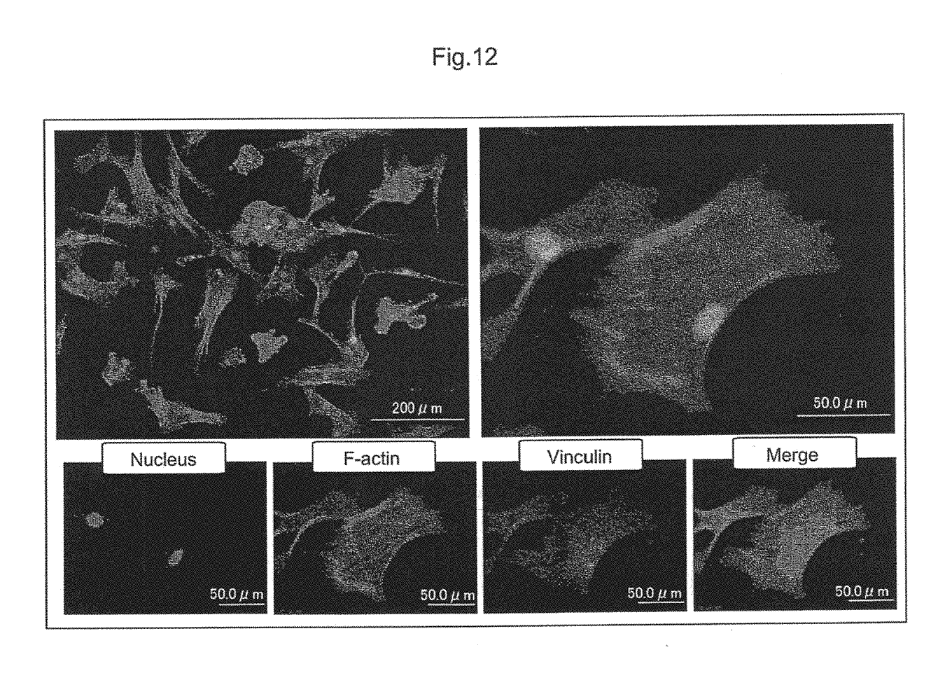

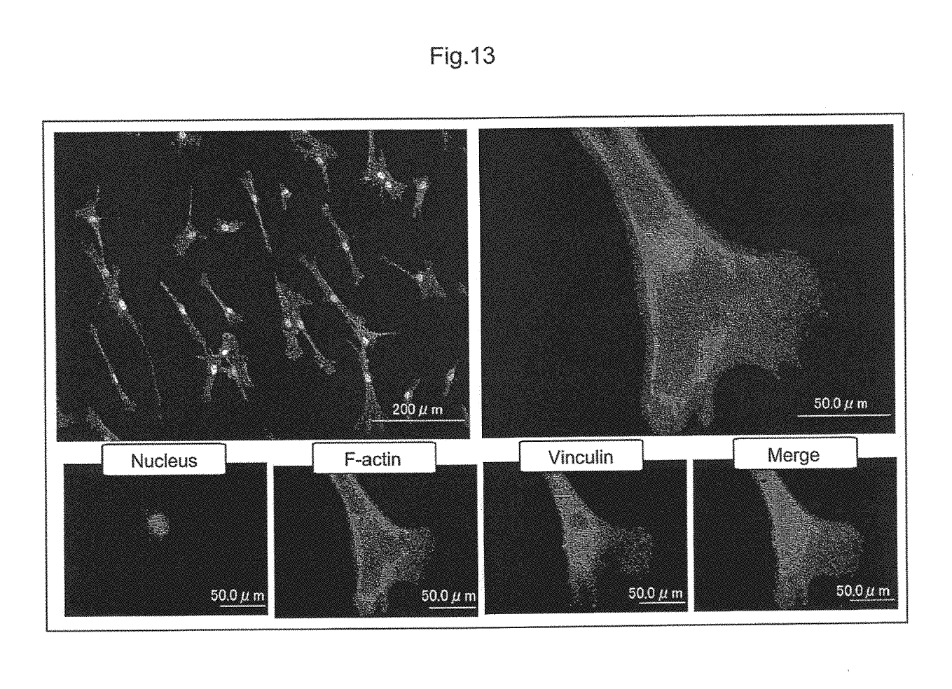

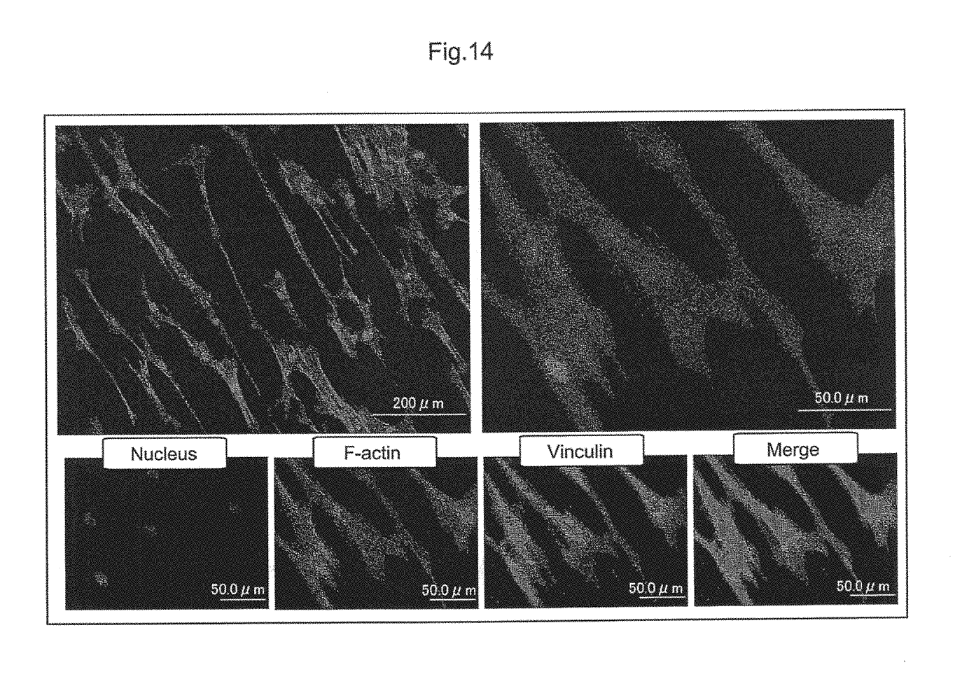

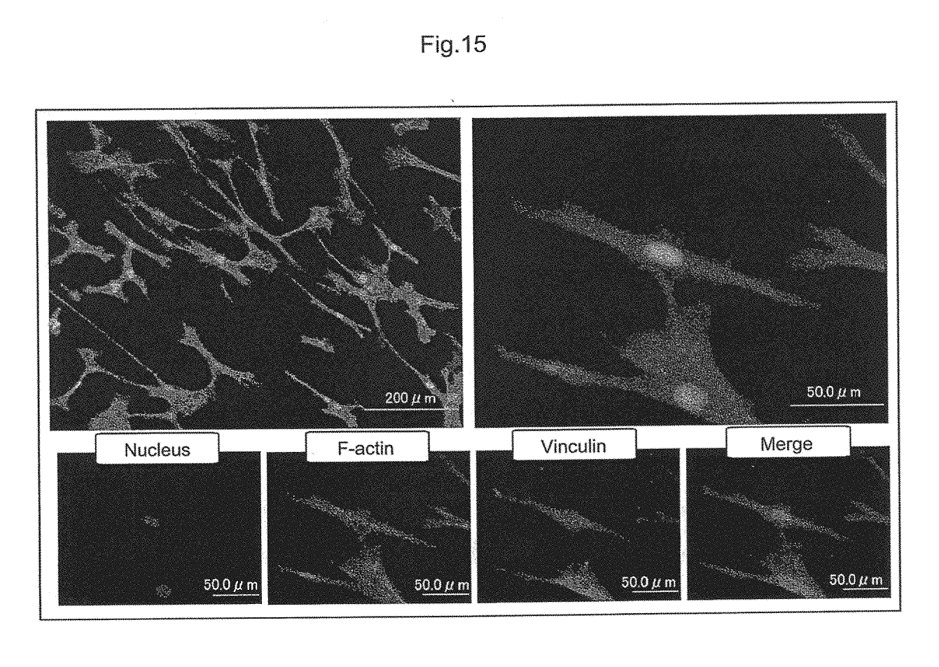

FIGS. 12 to 15 show the results. FIG. 12 shows the results from the control culture substrate (mirror surface), FIG. 13 shows the results from the culture substrate on which the periodic microgrooves were formed, FIG. 14 shows the results from the culture substrate 1 on which the hybrid periodic grooves 4a in which the periodic microgrooves 2a and the periodic nanogrooves 3a coexist were formed, and FIG. 15 shows the results from the culture substrate on which the periodic nanogrooves were formed. In the control, MSCs had a cell morphology having no directional property in particular, but it was found that, in the culture substrate on which the periodic microgrooves were formed, the culture substrate on which the periodic nanogrooves were formed, and the culture substrate 1 on which the hybrid periodic grooves 4a were formed, MSCs had a directional property and extended. It was found that, particularly in the culture substrate 1 on which the hybrid periodic grooves 4a were formed, MSCs extended specifically in one direction in an elongated manner. It can be understood from these results that MSCs are maintained in a state in which biological activity is high on the culture substrate 1 on which the hybrid periodic grooves 4a are formed, and thus the culture substrate 1 has higher biological cell affinity than the other culture substrates have. It is thought that the reason for this is that the fine structure of the culture substrate 1 of the present disclosure preferably fits to pseudopodia formed by the cytoskeletal molecules and provides favorable scaffolding for cell adhesion.

### Example 5

### Confirmation of Undifferentiation Property of MSCs on Culture Substrate

1. Summary

In this example, it was confirmed that MSCs seeded on the culture substrates in which the periodic fine structure was formed on the surface, the culture substrates being produced in Example 3 based on the processing conditions determined in Example 2 above, were maintained as undifferentiated MSCs. It is necessary to stably provide a high-quality stem cell population in order to provide differentiated cells in an amount sufficient enough to use the differentiated cells in regenerative medicine, a drug development screening, and the like. To achieve this, it is necessary to efficiently proliferate stem cells in a state in which the undifferentiation property is maintained.

It can be confirmed that MSCs seeded on the culture substrates are maintained as undifferentiated MSCs by confirming the cell morphology of the MSCs, the expression of a MSC-specific surface antigen, and the ability to differentiate (e.g., differentiate into osteocytes, chondrobrasts, and the like). In this example, the expression of the MSC-specific surface antigen was confirmed.

2. Testing Method

It was confirmed whether or not MSCs cultured on the above-mentioned culture substrates were maintained in the undifferentiated state by confirming the expression of MSC-specific surface antigens. MSCs exhibit a CD44-positive (CD44⁺) phenotype, a CO73-positive (CD73⁺) phenotype, a CD90-positive (CD90⁺) phenotype, a CD105-positive (CD105⁺) phenotype, a CD34-negative (CD34⁻) phenotype, and a C45-negative (CD45⁻) phenotype. Therefore, it was confirmed whether or not MSCs cultured on the culture substrates exhibited the above-mentioned phenotypes. TCPS was also used to perform the same treatment, and the phenotypes of the specific surface antigens were confirmed.

MSCs were seeded on the culture substrates and cultured for 48 hours in the same manner as in Example 3. After the culturing, expressions of cell surface antigens CD44, CD73, CD90, CD105, CD34, and CD45 were analyzed. At this time, MSCs cultured on tissue culture polystyrene (TCPS), which is confirmed to enable the undifferentiation property of MSCs that is to be maintained, were used to analyze the expression of the above-mentioned cell surface antigens in the same manner.

The expression of the cell surface antigens were analyzed using a real time reverse transcription polymerase chain reaction (real time RT-PCR) gene analysis method. Specifically, total RNA was extracted from the cells after the culturing, and cDNA was synthesized from the RNA through a reverse transcription reaction.

Subsequently, a PCR was performed using the synthesized cDNA as a template. With this method, a plurality of types of genes can be detected with a PCR using the syntesized cDNA by selecting optimum reverse transcription primers according to the purpose of the experiment. Since gene-specific primers are used in the reverse transcription reaction, specific genes can be detected with high sensitivity.

Here, PrimerBank of Harvard Medical School in the U.S. (http://pga.mgh.harvard.edu/primerbank/) was used to design gene-specific primers. Greiner Bio-One was requested to produce the designed primers. The sequence data of the gene-specific primers used in this example were summarized in Table 3. Moreover, the expression level of GAPDH was measured as a control, and the expression levels of the phenotypes were calculated using the expression level of GAPDH as an Index.

TABLE 3

| | | Sequence of gene-specfic | |
|---|---|---|---|
| | Phenotype | primer (5′→3′) | |
| Positive | CD44 | Forward | CTGCCGCTTTGCAGGTGTA |
| | | | (Sequence ID No. 1) |
| | | Reverse | CATTGTGGGCAAGGTGCTATT |
| | | | (Sequence ID No. 2) |
| | CD73 | Forward | GCCTGGGAGCTTACGATTTTG |
| | | | (Sequence ID No. 3) |
| | | Reverse | TAGTGCCCTGGTACTGGTCG |
| | | | (Sequence ID No. 4) |
| | CD90 | Forward | ATCGCTCTCCTGCTAACAGTC |
| | | | (Sequence ID No. 5) |
| | | Reverse | CTCGTACTGGATGGGTGAACT |
| | | | (Sequence ID No. 6) |
| | CD105 | Forward | TGCACTTGGCCTACAATTCCA |
| | | | (Sequence ID No. 7) |
| | | Reverse | AGCTGCCCACTCAAGGATCT |
| | | | (Sequence ID No. 8) |
| Negative | CD34 | Forward | CTACAACACCTAGTACCCTTGGA |
| | | | (Sequence ID No. 9) |
| | | Reverse | GGTGAACACTGTGCTGATTACA |
| | | | (Sequence ID No. 10) |
| | CD45 | Forward | ACCACAAGTTTACTAACGCAAGT |
| | | | (Sequence ID No. 11) |
| | | Reverse | TTTGAGGGGGATTCCAGGTAAT |
| | | | (Sequence ID No. 12) |
| Control | GAPDH | Forward | GGAGCGAGATCCCTCCAAAAT |
| | | | (Sequence ID No. 13) |
| | | Reverse | GGCTGTTGTCATACTTCTCATGG |
| | | | (Sequence ID No. 14) |

3. Results

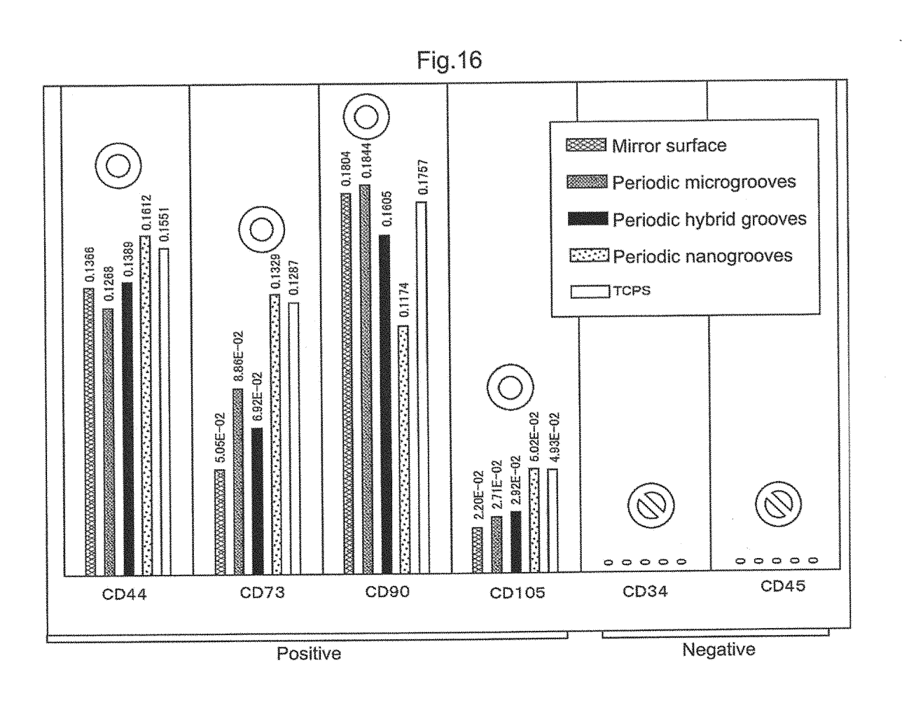

The graph in FIG. 16 shows the results. It was confirmed that all of the MSCs cultured on the control culture substrate (mirror surface), the culture substrate on which the periodic microgrooves were formed, the culture substrate on which the periodic nanogrooves were formed, and the culture substrate 1 on which the hybrid periodic grooves 4a in which the periodic microgrooves 2a and the periodic nanogrooves 3a coexist were formed exhibited the MSC-specific phenotypes including CD44⁺, CD73⁺, CD90⁺, CD105⁺, CD34⁻, and CD45⁻, similarly to the case of using TCPS. It can be understood that MSCs are maintained in the undifferentiated state on any of the culture substrates.

### Example 6

### Analysis of Induction of Differentiation of MSCs on Culture Substrate (Confirmation Using Differentiation Marker)

1. Summary

In this example, the induction of the differentiation of MSCs on the culture substrates in which the periodic fine structure was formed on the surface, the culture substrates being produced in Example 3 based on the processing conditions determined in Example 2 above, was examined. In this example, the induction of the differentiation of MSCs into osteocytes and chondrocytes was examined, and the differentiation induced state was confirmed using differentiation markers.

2. Testing Method

2-1. Induction of Differentiation

MSCs cultured on the above-mentioned culture substrates were cultured in a growth medium for 72 hours (until the confluence reached the above-mentioned desired level (100% in the case of the differentiation into osteocytes and chondrocytes)), and then cultured in a differentiation induction medium for 72 hours to induce the differentiation.

Here, the culture medium in Example 3 was used as the growth medium, and the differentiation of fourth-passage MSCs was induced. The following are the details of a differentiation inducing method.

2-1-1. Induction of Differentiation into Osteocytes

When MSCs are differentiated into osteocytes, the induction of the differentiation is started at the time when the confluence of MSCs reaches preferably 100%. hMSC-BulletKit™-osteogenic (Lonza, Catalog No. PT-3002) can be used as a culture medium for the induction of differentiation into osteocytes to induce the differentiation into osteocytes in accordance with the instructions of the manufacturer. This kit includes a basal medium, L-glutamine, dexamethasone, ascorbic acid, ITS+supplement (included in hMSC-BulletKit™-chondrogenic (Lonza. Catalog No. PT-3003)) sodium pyruvate, proline, a mesenchymal cell growth supplement (MCGS). β-glycerophosphate, and penicillin/streptomycin. It is preferable that the initial cell seeding density is 3.1×10⁵ cells/cm².

2-1-2. Induction of Differentiation into Chondrocytes

When MSCs are differentiated into chondrocytes, the induction of the differentiation is started at the time when the confluence of MSCs reaches preferably 100%. hMSC-BulletKit™-chondrogenic (Lonza, Catalog No. PT-3003) can be used as a culture medium for the induction of differentiation into chondrocytes to induce the differentiation into chondrocytes in accordance with the instructions of the manufacturer.

This kit includes a basal medium, L-glutamine, dexamethasone, ascorbic acid, ITS+supplement, sodium pyruvate, proline, and GA-1000 (gentamicin, amphotericin 8). It is preferable that the initial cell seeding density is 5×10⁵ cells/cm².

2-2. Confirmation of Induction of Differentiation

After the culturing, the induction of the differentiation was confirmed by analyzing differentiation markers that are not expressed in MSCs but are expressed specifically in osteocytes and chondrocytes. The differentiation into osteocytes was confirmed using SPP1 as the differentiation marker, and the differentiation into chondrocytes was confirmed using SOX9 as the differentiation marker. N=3 in the experiments.

The expression of the cell differentiation markers was analyzed using a real time RT-PCR gene analysis method in the same manner as in Example 5. Specifically, total RNA was extracted from the cells after the induction of the differentiation, and cDNA was synthesized from the RNA through a reverse transcription reaction. Subsequently, a PCR was performed using the synthesized cDNA as a template.

Here, PrimerBank of Harvard Medical School in the U.S. (http://pga.mgh.harvard.edu/primerbank/) was used to design gene-specific primers. Greiner Bio-One was requested to produce the designed primers. The sequence data of the gene-specific primers used in this example were summarized in Table 4. Moreover, the expression level of GAPDH was measured as a control in the same manner as in Example 5.

TABLE 4

| Marker | Gene | Sequence of gene-specific primer (5′→3′) | | |
|---|---|---|---|---|
| Differentiation | SPP1 | Forward | CTCCATTGACTCGAACGACTC | (Sequence ID |
| into | | | | No. 15) |
| osteocytes | | Reverse | CAGGTCTGCGAAACTTCTTAGAT | (Sequence ID |
| | | | | No. 16) |
| Differentiation | SOX9 | Forward | AGCGAACGCACATCAAGAC | (Sequence ID |
| into | | | | No. 17) |
| chondrocytes | | Reverse | CTGTAGGCGATCTGTTGGGG | (Sequence ID |
| | | | | No. 18) |
| Control | GAPDH | Forward | GGAGCGAGATCCCTCCAAAAT | (Sequence ID |
| | | | | No. 13) |
| Control | GAPDH | Reverse | GGCTGTTGTCATACTTCTCATGG | (Sequence ID |
| | | | | No. 14) |

3. Results

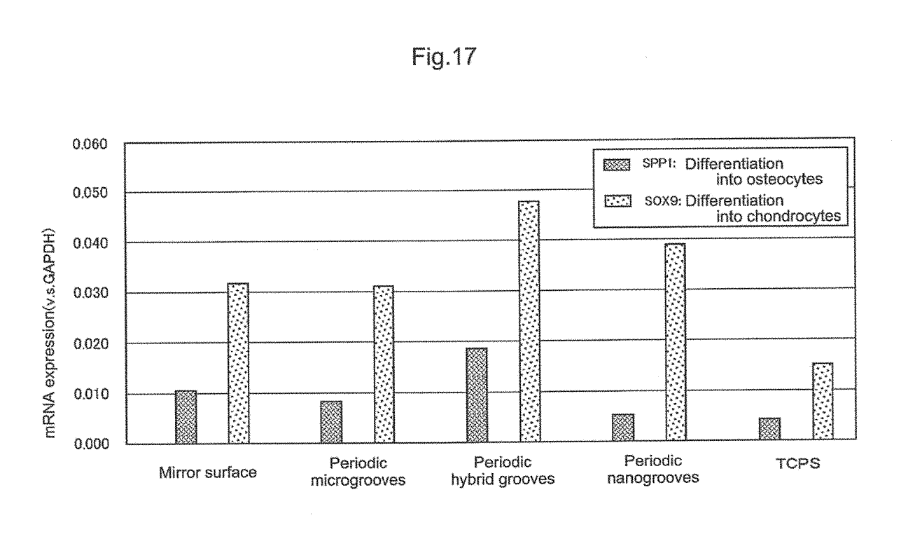

The graph in FIG. 17 shows the results. As a result, regarding the differentiation of MSCs into osteocytes and chondrocytes, the expression levels of the differentiation markers in the case of the culture substrate 1 on which the hybrid periodic grooves 4a were formed were higher than those in the cases of the control culture substrate and the other culture substrates including the culture substrate on which the periodic microgrooves were formed and the culture substrates on which the periodic nanogrooves were formed. It can be understood from these results that the hybrid periodic grooves 4a promote the ability of MSCs to be differentiated into osteocytes and chondrocytes.

### Example 7

### Induction of Differentiation of MSCs on Culture Substrate (Confirmation by Observation Under Fluorescent Microscope)

1. Summary

Subsequent to Example 6, in this example, the induction of the differentiation of MSCs on the culture substrates in which the periodic fine structure was formed on the surface, the culture substrates being produced in Example 3 based on the processing conditions determined in Example 2 above, was examined. In this example, the induction of the differentiation of MSCs into osteocytes and chondrocytes was examined, and the differentiation induced state was confirmed by observing the cell morphology under a fluorescent microscope.

2. Testing Method

2-1. Induction of Differentiation

MSCs cultured on the above-mentioned culture substrates were cultured in a growth medium for 72 hours (until the confluence reached the above-mentioned desired level (100% in the case of the differentiation into osteocytes and chondrocytes)), and then cultured in a differentiation induction medium for 48 hours to induce the differentiation in the same manner as in Example 6.

2-2. Confirmation of Induction of Differentiation

After the culturing, the induction of the differentiation was confirmed by observing the cell morphology under a fluorescent microscope. The cell morphology of cells cultured only in a growth medium without inducing the differentiation was also observed under a fluorescent microscope in the same manner.

3. Results

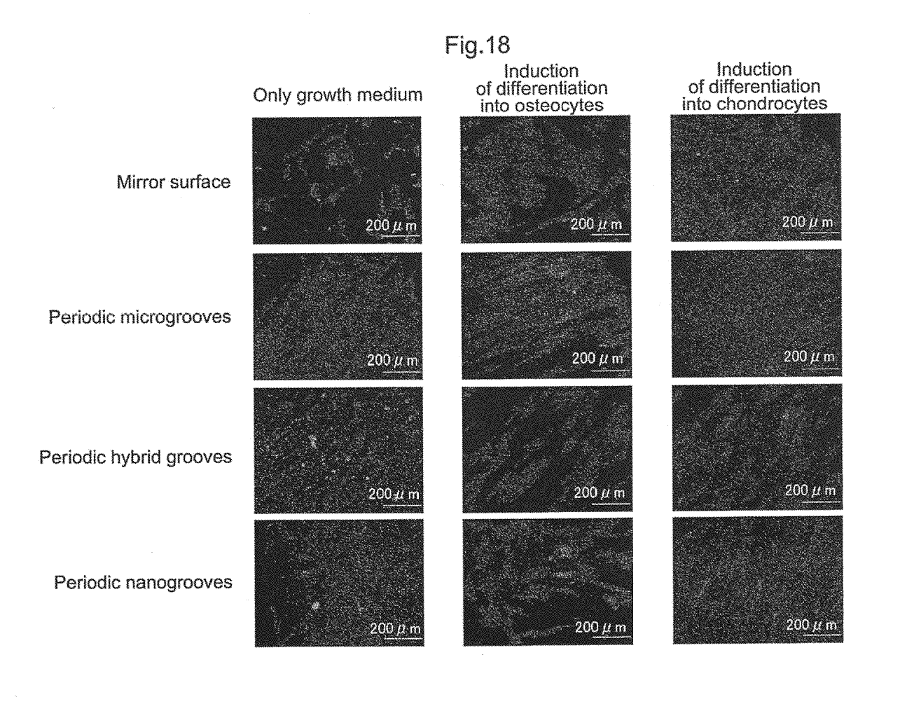

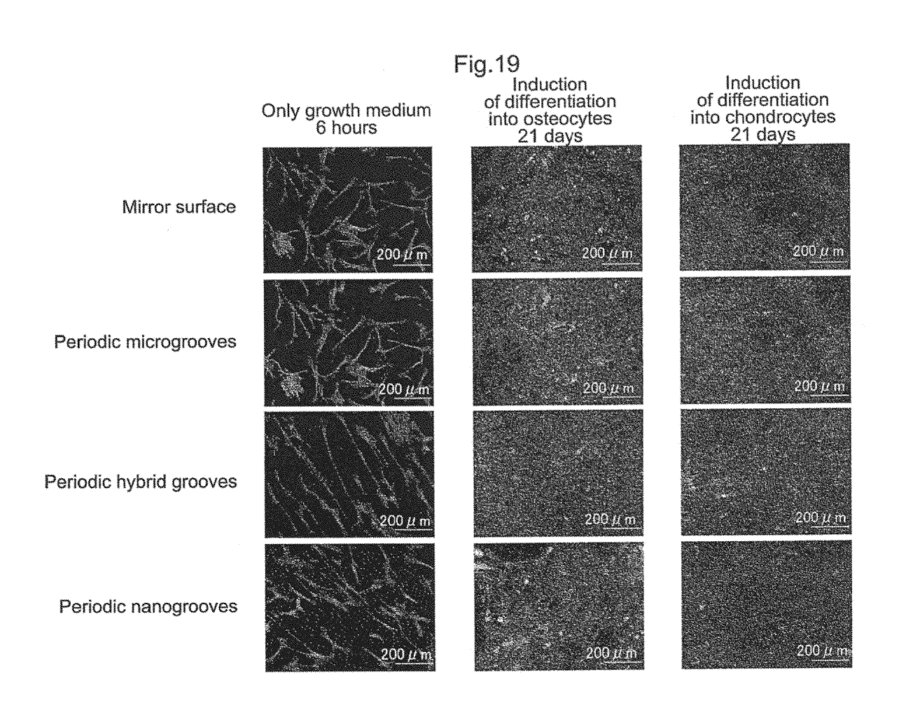

FIG. 18 shows the results. Compared with the case in which the culturing was performed using only the growth medium, in the cases where the culturing was performed using the culture medium for the induction of differentiation into osteocytes and the culturing was performed using the culture medium for the induction of differentiation into chondrocytes, it was confirmed that the cell morphology changed in all the cases including the case of the control, the case of the culture substrate on which the periodic microgrooves were formed, the case of the culture substrate on which the periodic nanogrooves were formed, and the case of the culture substrate 1 on which the hybrid periodic grooves 4a were formed. It can be understood from these results that the differentiation of MSCs is induced.

### Example 8

### Analysis of Induction of Differentiation of MSCs on Culture Substrate (Confirmation by Immunofluorescence Staining)

1. Summary

Subsequent to Examples 6 and 7, in this example, the induction of the differentiation of MSCs on the culture substrates in which the periodic fine structure was formed on the surface, the culture substrates being produced in Example 3 based on the processing conditions determined in Example 2 above, was examined. In this example, the induction of the differentiation of MSCs into osteocytes and chondrocytes was examined and confirmed by performing immunofluorescence staining using a differentiation marker on the cells.

2. Testing Method

2-1. Induction of Differentiation

MSCs cultured on the above-mentioned culture substrates were cultured in a growth medium for 72 hours (until the confluence reached the above-mentioned desired level (100% in the case of the differentiation into osteocytes and chondrocytes)), and then cultured in a differentiation induction medium for a predetermined period of time to induce the differentiation in the same manner as in Example 5. The differentiation into osteocytes was induced by culturing the cells in the above-mentioned culture medium for the induction of differentiation into osteocytes for 14 to 21 days, and the differentiation into chondrocytes was induced by culturing the cells in the above-mentioned culture medium for the induction of differentiation into chondrocytes for 17 to 21 days.

2-2. Confirmation of Induction of Differentiation

After the culturing, the induction of the differentiation was confirmed by analyzing differentiation markers that were not expressed in MSCs but were expressed specifically in osteocytes and chondrocytes using an immunofluorescence staining method. The differentiation into osteocytes was confirmed using an anti-osteopontin antibody, and the differentiation into chondrocytes was confirmed using an anti-aggrecan antibody. The antibodies were detected by immunofluorescence staining using a secondary antibody.

The following fluorescent reagents were used.

a. Primary antibody

a-1. Antibody against osteocytes

Anti-Osteocalcin antibody (GENETEX, Inc., Catalog No. GTX39512)

a-2. Antibody against chondrocytes

Anti-Aggrecan, Rabbit-Poly <Anti-ACAN> (GENETEX, Inc., Catalog No. GTX113122)

b. Secondary antibody

Alexa Fluor (registered trademark) 488 goat-anti-mouse IgG (Alexa Fluor, Catalog No. A11001)

A specific detection method is as follows. A primary antibody staining solution was added in a sufficient amount to cover the cells on the culture substrates as samples and incubated at room temperature for one hour. After the reaction took place, the primary antibody staining solution was removed from the samples, and the samples were cleaned three times using PBS. Subsequently, a secondary antibody staining solution was added in a sufficient amount to cover the samples and incubated at room temperature for 30 minutes to one hour. After the reaction took place, the samples were cleaned three times using PBS, and the cells were observed under a fluorescent microscope.

Cells cultured only in a growth medium without inducing the differentiation was also subjected to fluorescent staining in the same manner and then observed under a fluorescent microscope.

3. Results

FIG. 19 shows the results. Compared with the case in which the culture was performed using only the growth medium, in the cases where the culture was performed using the culture medium for the induction of differentiation into osteocytes and the culture was performed using the culture medium for the induction of differentiation into chondrocytes, fluorescence derived from osteocytes and chondrocytes could be observed in all of the cases including the case of the control, the case of the culture substrate on which the periodic microgrooves were formed, the case of the culture substrate on which the periodic nanogrooves were formed, and the case of the culture substrate 1 on which the hybrid periodic grooves 4a were formed. In particular, it was confirmed that the induced cells increased particularly in the culture using the culture substrate 1 on which the hybrid periodic grooves 4a were formed.

### Example 9

### Evaluation of Induction of Differentiation of MSCs into Bone (Measurement of Alkaline Phosphatase Activity)

1. Summary

In this example, the induction of the differentiation of MSCs into bone was examined in detail based on the results of Examples 6 to 8. MSCs are differentiated into osteoblasts via precursor cells. In this differentiation process, alkaline phosphatase (referred to as “ALP” hereinafter) is expressed at an early stage, and then osteocalcin and the like, which is specific to bone, are expressed. In this example, the induction of the differentiation of MSCs into bone was evaluated by measuring the activity of ALP expressed at an early stage of the differentiation of precursor cells into osteoblasts.

2. Testing Method

MSCs cultured on the above-mentioned culture substrates were cultured in a growth medium for 72 hours (until the confluence reached the above-mentioned desired level (100% in the case of the differentiation into osteocytes)), and then cultured in a differentiation induction medium for 10 days to induce the differentiation of the MSCs into bone in the same manner as in Example 5 (N=2). The ALP activity was measured using LabAssay™ ALP kit (Wako Pure Chemical Industries, Ltd., Catalog No. PT-2501).

The measurement principle will be briefly described. When a specimen is reacted in a carbonate buffer (pH 9.8) containing p-nitrophenyl phosphate, ALP in the specimen decomposes the p-nitrophenyl phosphate into p-nitrophenol and phosphoric acid, and the produced p-nitrophenol is colored yellow on the alkali side. The activity value of ALP in the specimen can be determined by measuring the absorbance at 405 nm.

Specifically, after the induction of the differentiation, the culture medium in the well of the cell culture plate was aspirated, and the cells were cleaned twice using PBS. The cells were scraped up in 500 μl of ice-cold PBS, transferred to a microcentrifugation tube, and collected by using centrifugation (3000×g for 15 minutes). The supernatant was carefully removed, and the cells were resuspended in an ice-cold 50 mM Tris-HCl solution for ultrasonication to provide a cell suspension. This cell suspension was cooled on ice for 10 minutes. Subsequently, an operation in which the cell suspension was subjected to ultrasonication for 10 seconds while cooled on ice and then cooled for seconds was repeated about ten times. After the ultrasonication, cell residue was precipitated by using centrifugation (20,000×g for 20 minutes), and the soluble portion was used as a sample for the measurement of the ALP activity.

Then, 20 μL of the soluble portion was transferred into each well of a 96-well plate, and 100 μL of a substrate buffer (included in the kit) was dispensed into each well. Subsequently, the 96-well plate was shaken for about 1 minute, followed by incubation at 37° C. for 15 min immediately after that. Immediately after the incubation, 80 μL of a reaction stop solution (included in the kit) was dispensed into each well, and then the 96-well plate was shaken for 1 minute. The absorbance at 405 nm was measured using a microplate reader.

Enzyme activity for producing 1 nmol of p-nitrophenol at pH 9.8 at 37° C. for one minute was taken as one unit, and the ALP activity was calculated based on the following formula.

Activity(unit/μL)=C/15×a Formula 1
C: p-nitrophenol concentration (mmol/L=nmol/μL) against absorbance (A test-A blank), obtained from standard curve
15: reaction time (minute)
a: dilution ratio of specimen

TCPS was also used to perform the same treatment, and the ALP activity was measured.

3. Results

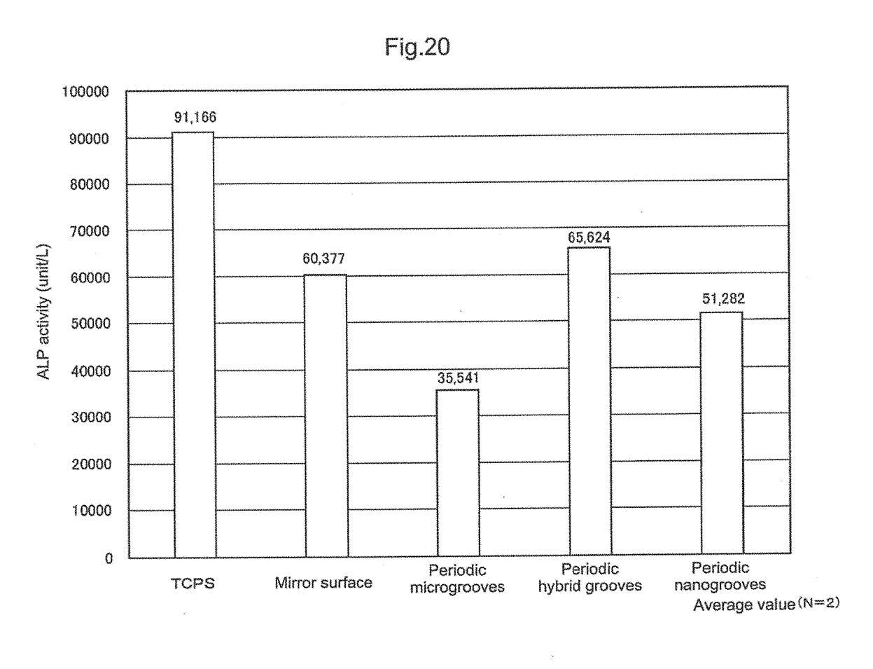

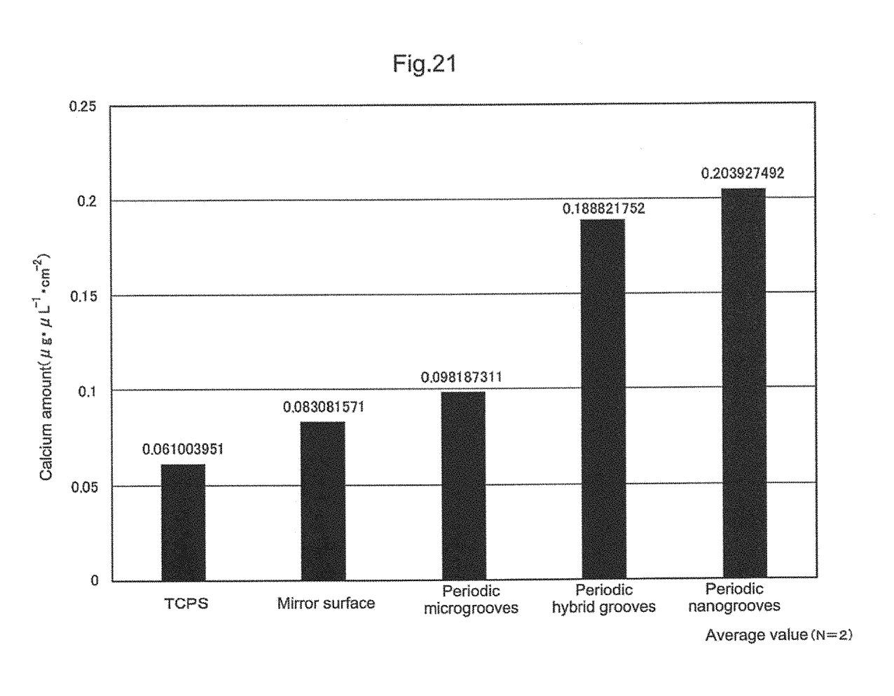

The graph in FIG. 20 shows the results.

The cells differentiated on the culture substrate 1 on which the hybrid periodic grooves 4a were formed had higher ALP activity than the cells differentiated on the control, the culture substrate on which the periodic microgrooves were formed, and the culture substrate on which the periodic nanogrooves were formed. Therefore, it can be understood that the culture substrate 1 on which the hybrid periodic grooves 4a are formed may promote the induction of the differentiation of MSCs into osteocytes.

### Example 10

### Evaluation of Induction of Differentiation of MSCs into Bone (Evaluation of Calcification Ability)

1. Summary

Subsequent to Example 9, in this example, the induction of the differentiation of MSCs into bone was examined in detail. Although the induction of the differentiation was evaluated using the ALP activity in Example 9, ALP is expressed at a high level at an early stage of the differentiation of precursor cells into osteoblasts as mentioned above. When the differentiation into osteoblasts and subsequent osteocytes progresses, the ALP activity decreased. In Example 10, the evaluation based on calcification ability was also performed. Osteoblasts are cells that play a role in forming bone, and induce the synthesis of bone matrix proteins and the calcification via matrix vesicles. On the other hand, osteoblasts become embedded in the bone matrix, which has been produced by osteoblasts themselves, and are differentiated into osteocytes.

2. Testing Method

MSCs cultured on the above-mentioned culture substrates were cultured in a growth medium for 72 hours (until the confluence reached the above-mentioned desired level (100% in the case of the differentiation into osteocytes)), and then cultured in a differentiation induction medium for 10 days to induce the differentiation of the MSCs into bone in the same manner as in Example 5 (N=2). The calcification ability was evaluated by measuring the amount of calcium.

The amount of calcium was measured using a Calcium Colorimetric Assay Kit (BioVision Inc., Catalog No. K380-250). Specifically, after the induction of the differentiation, the culture medium in the well of the cell culture plate was aspirated, and the cells were cleaned twice using PBS. The cells were scraped up in 500 μl of ice-cold PBS, transferred to a microcentrifugation tube, and collected by using centrifugation (3000×g for 15 minutes). The supernatant was carefully removed, and the cells were resuspended in an ice-cold 50 mM Tris-HCl solution for ultrasonication to provide a cell suspension. This cell suspension was cooled on ice for 10 minutes. Subsequently, an operation in which the cell suspension was subjected to ultrasonication for 10 seconds while cooled on ice and then cooled for 20 seconds was repeated about ten times. After the ultrasonication, cell residue was precipitated by using centrifugation (20,000×g for 20 minutes), and the soluble portion was used as a sample for the measurement of the amount of calcium.

Then, 50 μL of the soluble portion was transferred into each well of a 96-well plate, and 90 μL of Chromogenic Reagent (included in the kit) was dispensed into each well. Then, the 96-well plate was shaken. Subsequently, 60 μL of Calcium Assay Buffer (included in the kit) was dispensed, and the 96-well plate was shaken. The plate was shielded from light and incubated at room temperature for 10 minutes, and the absorbance at 575 nm was measured using a microplate reader.

The amount of calcium was calculated as calcium amount (μg/μL) against absorbance (A test-A blank), obtained from a standard curve (reagents for preparing a standard curve are included in the kit).

TCPS was also used to perform the same treatment, and the amount of calcium was measured.

3. Results

The graph in FIG. 21 shows the results. The cells differentiated on the culture substrate on which the periodic nanogrooves were formed and the culture substrate 1 on which the hybrid periodic grooves 4a were formed contained calcium in a larger amount than the cells differentiated on the control and the culture substrate on which the periodic microgrooves were formed. Therefore, it can be understood that the culture substrate 1 on which the hybrid periodic grooves 4a are formed and the culture substrate on which the periodic nanogrooves are formed may promote the calcification and the induction of the differentiation of MSCs into osteocytes.

II. Examination Using Culture Substrate 1 on which Hybrid Periodic Lattice Grooves+Periodic Projections 4b are Formed

### Example 11

### Examination of Production of Culture Substrate 1

1. Summary

In this example, in order to further examine the relationship between the effect of inducing the differentiation of MSCs and the fine surface structure of the culture substrate 1, the production of a culture substrate 1 in which a periodic fine structure different from those formed on the culture substrates 1 examined in Examples 1 to 10 in section I above was formed on the surface was examined.

2. Materials and Methods

2-1. Culture Substrate

A medical titanium plate (914 mm×1 mm) in which one side was polished into a mirror surface was used as a substrate, and culture substrates 1 in which the periodic fine structure described below were formed on the substrate surface were produced using a femto-second laser D-1000. The femto-second laser D-1000 used here was the same as that used in Example 1, and the output was 1.2 W, the pulse width was 400 fs, and the repetition frequency was 100 kHz.

2-1-1. Culture Substrate in which Periodic Lattice Grooves in the Order of Micrometers are Formed on Substrate Surface (Comparative Example)

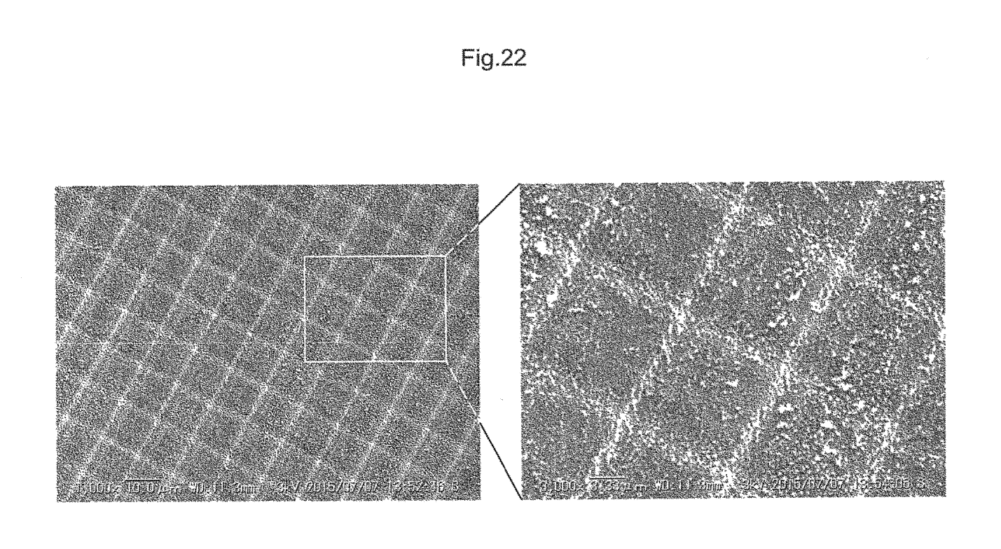

Periodic lattice grooves in the order of micrometers (which may be referred to as “periodic lattice microgrooves” hereinafter) were formed by irradiating and scanning longitudinally and horizontally the surface of the above-mentioned medical titanium plate with a femto-second laser beam (fluence: 0.7 J/cm², scanning speed: 300 mm/second, scanning frequency: 14 times, polarized light circularly polarized light). As a result, the periodic lattice grooves having a width of 6 μm, a depth of 0.6 μm, and a pitch of 12 μm were formed (1). FIG. 22 shows a scanning electron microscope (SEM) image of the processed surface of the culture substrate in which the periodic lattice microgrooves were formed on the substrate surface. It should be noted that rough surfaces located in the region surrounded by the lattice grooves are not processing traces but debris generated during laser processing.

2-1-2. Culture Substrate in which Periodic Microgrooves are Formed on Substrate Surface (Comparative Example)

Periodic microgrooves were formed by irradiating and scanning the surface of the above-mentioned medical titanium plate with a femto-second laser beam (fluence: 0.7 J/cm², scanning speed: 300 mm/second, scanning frequency: 14 times, polarized light circularly polarized light). As a result, the periodic grooves having a width of 6 μm, a depth of 0.6 μm, and a pitch of 12 μm were formed (2).

2-1-3. Culture Substrate in which Periodic Nanogrooves are Formed on Substrate Surface (Comparative Example)

Periodic nanogrooves were formed by irradiating and scanning the surface of the above-mentioned medical titanium plate with a femtosecond laser beam (fluence: 0.8 J/cm², scanning speed: 500 mm/second, scanning frequency: once, polarized light: linearly polarized light). As a result, the periodic grooves having a depth of 0.2 μm and a pitch of 0.7 μm were formed (3).

2-1-4. Culture Substrate 1 in which Hybrid Periodic Lattice Grooves+Periodic Projections 4b are Formed on Substrate Surface (Example)

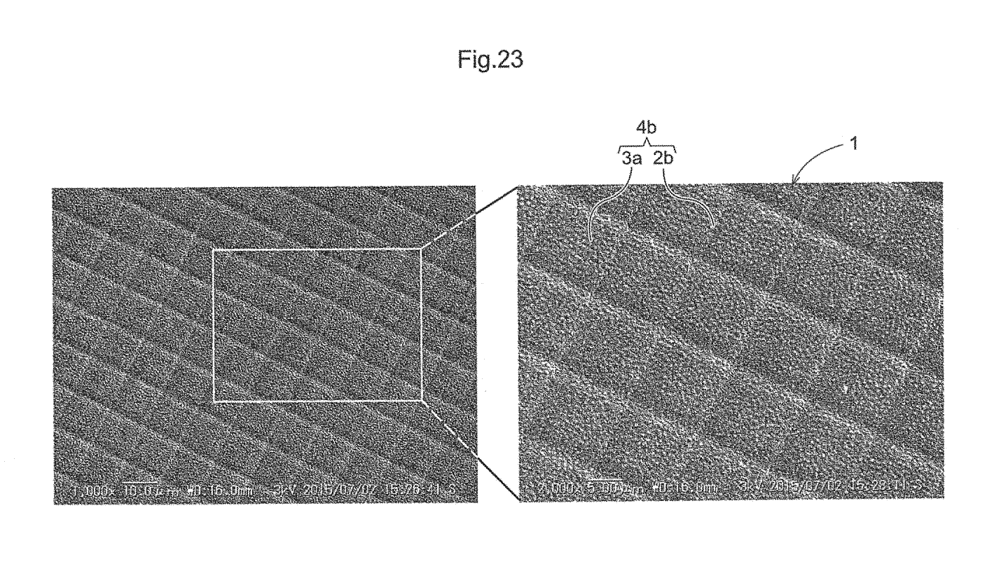

This culture substrate 1 was a culture substrate 1 in which hybrid periodic lattice grooves+periodic projections 4b in which periodic lattice microgrooves 2b and periodic projections 3b in the order of nanometers (which may be referred to as “periodic nanoprojections” hereinafter) coexist were formed on the substrate surface, and were formed by irradiating and scanning the surface of the above-mentioned medical titanium plate with a femto-second laser beam. First, the periodic lattice microgrooves 2b were formed in accordance with the procedure described in 2-1-1, above except that the scanning frequency was 20 times, and then the periodic nanoprojections 3b were formed to overlap the periodic lattice microgrooves 2b. The periodic nanoprojections 3b were formed in the conditions in which the fluence was 0.8 J/cm², the scanning speed was 500 mm/second, the scanning frequency was once, and the polarized light was circularly polarized light. As a result, the culture substrate 1 having the hybrid periodic lattice grooves+periodic projections 4b in which the periodic lattice microgrooves 2b having a groove width of 6 μm, a depth of 0.6 μm, and a pitch of 12 μm, and the periodic nanoprojections 3b having a diameter of 0.6 μm, a height of 0.2 μm, and a pitch of 0.7 μm were formed was produced (4). FIG. 23 shows a scanning electron microscope (SEM) image of the processed surface of the produced culture substrate in which the hybrid periodic lattice grooves+periodic projections 4b are formed on the substrate surface.

2-1-5. Culture Substrate 1 in which Hybrid Periodic Grooves 4a (Periodic Microgrooves 2a+Periodic Nanogrooves 3a) were Formed on Substrate Surface Example

This culture substrate was a culture substrate (FIGS. 7 to 10) in which the hybrid periodic grooves 4a in which the periodic microgrooves and the periodic nanogrooves described in section I above coexist were formed on the substrate surface, and were formed by irradiating and scanning the surface of the above-mentioned medical titanium plate with a femto-second laser beam. First, the periodic microgrooves 2a were formed in accordance with the procedure described in 2-1-2, above except that the scanning frequency was 20 times, and then the periodic nanogrooves 3a were formed to overlap the periodic microgrooves 2a in accordance with the procedure described in 2-1-3, above. In the case of the periodic nanogrooves 3a, the orientation of the polarized light was orthogonal to the scanning direction. As a result, a culture substrate 1 having the hybrid periodic grooves 4a obtained by forming the periodic nanogrooves 3a having a depth of 0.2 μm and a pitch of 0.7 μm on the periodic microgrooves 2a having a groove width of 6 μm, a depth of 0.6 μm, and a pitch of 12 μm was produced (5).

### Example 12

### Analysis of Induction of Differentiation of MSCs on Culture Substrate (Confirmation Using Differentiation Marker)

1. Summary

In this example, the induction of the differentiation of MSCs on the culture substrates in which the periodic fine structure was formed on the surface, the culture substrates being produced in Example 11 above, was examined. In this example, the induction of the differentiation of MSCs into osteocytes, chondrocytes, nerve cells, and adipocytes was examined, and the differentiation induced state was confirmed using differentiation markers.

2. Materials and Testing Method

2-1. Culture Substrate

The culture substrates of comparative examples and examples produced in 2-1-1, to 2-1-5, in Example 11 above were used. A mirror-polished medical titanium plate that was used as it was without being subjected to laser processing was taken as a control (6).

2-2. Cells

Human MSCs (hMSCs, mesenchymal stem cells, Lonza, Catalog No. PT-2501) were used.

2-3. Induction of Differentiation

Each of the above-mentioned culture substrates were immersed in 70% ethanol for 20 minutes to be sterilized, and then cleaned three times using distilled water. After the cleaning, the culture substrate was left to stand on the bottom surface of a well of a 12-well cell culture plate, a culture medium was poured into the well, and thus the culture substrate was immersed in the culture medium. MSCs were seeded on each of the culture substrates immersed in the culture medium and cultured for six hours.

At this time, the initial cell seeding density was 5000 cells/cm². MSCGM™ BulletKit™ (Lonza, Catalog No. PT-3001), which had been prepared for use, was used as the culture medium.

MSCs cultured on the above-mentioned culture substrates were cultured in a growth medium for 72 hours (until the confluence reached the above-mentioned desired level (100% in the case of the differentiation into osteocytes and chondrocytes, and 80 to 90% in the case of the differentiation into nerve cells and adipocytes)), and then cultured in a differentiation induction medium for 72 hours to induce the differentiation. Here, the culture medium in Example 3 was used as the growth medium, and the differentiation of fourth-passage MSCs was induced. The following are the details of a differentiation inducing method.

2-1-1. Induction of Differentiation into Osteocytes

The differentiation into osteocytes was performed based on the method in Example 6.

2-1-2. Induction of Differentiation into Chondrocytes

The differentiation into chondrocytes was performed based on the method in Example 6.

2-1-3. Differentiation into Nerve Cells

When MSCs are differentiated into nerve cells, the induction of the differentiation can be started at the time when the confluence of MSCs reaches preferably 80 to 90%.

Mesenchymal Stem Cell Neurogenic Differentiation Medium (PromoCell, Catalog No. C-28015) can be used as a culture medium for the induction of differentiation into nerve cells to induce the differentiation into nerve cells in accordance with the instructions of the manufacturer. This kit includes a basal medium and Supplement Mix (PromoCell, Catalog No. C-39815). It is preferable that the initial cell seeding density is 5000 cells/cm².

2-1-4. Differentiation into Adipocytes

When the differentiation of MSCs into adipocytes is induced, the induction of the differentiation can be started at the time when the confluence of MSCs reaches preferably 80 to 90%. hMSC-BulletKit™-adipogenic (Lonza, Catalog No. PT-3004) can be used as a culture medium for the induction of differentiation into adipocytes to induce the differentiation into adipocytes in accordance with the instructions of the manufacturer. This kit includes a basal medium, L-glutamine, a mesenchymal cell growth supplement (MCGS), dexamethasone, indomethacin, 3-isobutyl-1-methylxanthine (IBMX), and GA-1000 (gentamicin, amphotericin B). It is preferable that the initial cell seeding density is 2.1×10⁴ cells/cm².

2-2. Confirmation of Induction of Differentiation

After the culturing, the induction of the differentiation was confirmed by analyzing differentiation markers that are not expressed in MSCs but are expressed specifically in osteocytes, chondrocytes, nerve cells, and adipocytes. The differentiation into osteocytes was confirmed using SPP1 as the differentiation marker, the differentiation into chondrocytes was confirmed using SOX9 as the differentiation marker, the differentiation into nerve cells was confirmed using MAP2 as the differentiation marker, and the differentiation into adipocytes was confirmed using PPARG as the differentiation marker. N=7 in the experiments.

The expression of the cell differentiation markers were analyzed using a real time RT-PCR gene analysis method. Specifically, total RNA was extracted from the cells after the induction of the differentiation, and cDNA was synthesized from the RNA through a reverse transcription reaction. Subsequently, a PCR was performed using the synthesized cDNA as a template. The expression level of GAPDH was measured in the same manner, and the relative expression level to the expression level of GAPDH was calculated. Moreover, in the case of tissue culture polystyrene (TCPS (7)), the induction of the differentiation into the aforementioned cells was confirmed. The sequence data of the gene-specific primers used in this example were summarized in Table 5. It should be noted that the gene specific primers mentioned in Table 4 in Example 6 were used as the gene specific primers for SPP1 for the confirmation of the differentiation into osteocytes, SOX9 for the confirmation of the differentiation into chondrocyte, and GAPDH.

TABLE 5

| Marker | Gene | Sequence of gene-specific primer (5′→3′) | | |
|---|---|---|---|---|
| Differentiation | MAP2 | Forward | CACTGGCGGTGCAACAAGA | (Sequence ID |
| into nerve cells | | | | No. 19) |
| | | Reverse | TTTCATAACAGCGGAGGCATTTC | (Sequence ID |
| | | | | No. 20) |
| Differentiation | PPARG | Forward | GCTGGACGTCCTGGTGAAG | (Sequence ID |
| into adipocytes | | | | No. 21) |
| | | Reverse | ACGTTGTCCAGCAATACCCTGAG | (Sequence ID |
| | | | | No. 22) |

3. Results

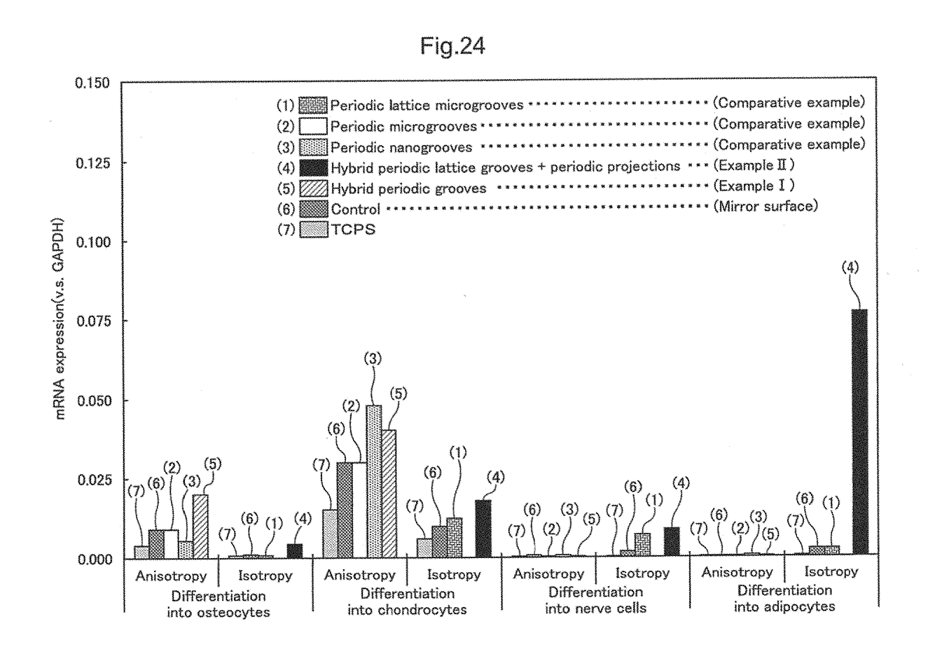

FIG. 24 shows the results. In this figure, (1) indicates the culture substrate of the comparative example in which the periodic lattice microgrooves are formed on the substrate surface, (2) indicates the culture substrate of the comparative example in which the periodic microgrooves are formed on the substrate surface, (3) indicates the culture substrate of the comparative example in which the periodic nanogrooves are formed on the substrate surface, (4) indicates the culture substrate of Example (II) in which the hybrid periodic lattice grooves+periodic projections 4b are formed on the substrate surface, (5) Indicates the culture substrate of Example (I) in which the hybrid periodic grooves 4a are formed on the substrate surface, (6) indicates the control (mirror surface), and (7) indicates TCPS.

In the case of the culture substrate 1 in which the hybrid periodic lattice grooves+periodic projections 4b are formed on the substrate surface, the expression of the differentiation marker for adipocytes was high, and a high accelerating effect on the induction of the differentiation into adipocytes was exhibited (4). Also, in the case of the culture substrate 1 in which the hybrid periodic lattice grooves+periodic projections 4b are formed on the substrate surface, an accelerating effect on the induction of the differentiation into nerve cells was exhibited (4). On the other hand, in the case of the culture substrate 1 in which the hybrid periodic grooves 4a are formed on the substrate surface, as confirmed in section I above, a high accelerating effect on the induction of the differentiation into osteocytes and chondrocytes was confirmed (5). In the cases where the periodic lattice microgrooves, the periodic microgrooves, and the periodic nanogrooves are individually formed on the substrate surface, and in the case of the control, there was no significant difference in the degree of differentiation from the case where the cells were cultured in an environment without a substrate, and it could not be said that an effect of accelerating the induction of the differentiation was exhibited (1, 2, 3, 6, 7).

It can be understood from these results that the hybrid periodic grooves 4a can exhibit a significant accelerating effect on the induction of the differentiation into bone and chondrocytes, whereas the hybrid periodic lattice grooves+periodic projections 4b can exhibit a significant accelerating effect on the induction of the differentiation into adipocytes and nerve cells. Accordingly, it can be understood that the periodic fine structure formed on the surface of the culture substrate closely relates to the induction direction of the differentiation.

In this manner, the periodic fine structure 2 in the order of micrometers and the periodic fine structure 3 in the order of nanometers are simultaneously formed on the surface of the culture substrate 1 of the present disclosure. When the two periodic fine structures are formed individually, the biocompatibility and the biological cell affinity are slightly improved, whereas when the two periodic fine structures coexist, the biocompatibility and the biological cell affinity are particularly improved. Moreover, with the culture substrate 1 of the present disclosure, stem cells can be efficiently proliferated in a state in which the undifferentiation property is maintained, thus making it possible to stably provide a high-quality stem cell population. Accordingly, the culture substrate 1 of the present disclosure can contribute to the development of techniques using stem cells such as those in regenerative medicine and drug development screening, which require a large amount of high-quality stem cells.

The culture substrate 1 of the present disclosure can be used in any field that particularly requires the culturing of stem cells, and particularly in industrial fields such as drug development, life science, and medical treatment. The culture substrate 1 of the present disclosure can be applied to a pharmacological test and a drug development screening in which the efficacy, pharmacokinetics, safety, and the like of a development candidate drug are evaluated; the clarification of a development mechanism, a differentiation mechanism, and a disease mechanism; and regenerative medicine and cell therapy in which the functions of impaired viscera and organs are regenerated, for example.

With the above-mentioned embodiments, the following configurations are evoked.

For example, in the above-mentioned embodiments, in the culture substrate having a periodic fine structure in the order of micrometers and a periodic fine structure in the order of nanometers on the same surface where stem cells are to be cultured on the surface, the periodic fine structure in the order of micrometers and the periodic fine structure in the order of nanometers are formed as periodic grooves.

With the present disclosure, a culture substrate for culturing stem cells can be provided in which both the periodic fine structure in the order of micrometers and the periodic fine structure in the order of nanometers are formed as periodic grooves. The periodic grooves in the order of micrometers and the periodic grooves in the order of nanometers are simultaneously formed on the surface of the culture substrate of the present disclosure. Since these two types of periodic grooves coexist, the biocompatibility and the biological cell affinity can be further improved. Moreover, with the culture substrate of the present disclosure, stem cells can be efficiently proliferated in a state in which the undifferentiation property is maintained, thus making it possible to stably provide a high-quality stem cell population. Accordingly, the culture substrate of the present disclosure can contribute to the further development of techniques using stem cells such as those in regenerative medicine and drug development screening, which require a large amount of high-quality stem cells.

Furthermore, in the above-mentioned embodiments, the periodic grooves in the order of micrometers have a width of 1 to 20 μm, a depth of 0.3 to 2 μm, and a pitch of 1 to 100 μm, and the periodic grooves in the order of nanometers have a width of 0.1 to 1 μm, a depth of 0.01 to 0.5 μm, and a pitch of 0.1 to 1 μm.

With the present disclosure, a culture substrate for culturing stem cells can be provided in which both the periodic fine structure in the order of micrometers and the periodic fine structure in the order of nanometers are formed as periodic grooves, and the sizes of the two types of periodic grooves are optimized. The periodic grooves in the order of micrometers and the periodic grooves in the order of nanometers with a favorable size are simultaneously formed on the surface of the culture substrate of the present disclosure. Since these two types of periodic grooves with a specific size coexist, the biocompatibility and the biological cell affinity can be further improved. Moreover, with the culture substrate of the present disclosure, stem cells can be efficiently proliferated in a state in which the undifferentiation property is maintained, thus making it possible to stably provide a high-quality stem cell population. Accordingly, the culture substrate of the present disclosure can contribute to the further development of techniques using stem cells such as those in regenerative medicine and drug development screening, which require a large amount of high-quality stem cells.

Furthermore, in the above-mentioned embodiments, the periodic grooves in the order of micrometers and the periodic grooves in the order of nanometers are arranged in parallel.

With the present disclosure, a culture substrate for culturing stem cells can be provided in which both the periodic fine structure in the order of micrometers and the periodic fine structure in the order of nanometers are formed as periodic grooves, and the two types of periodic grooves are arranged in parallel. The periodic grooves in the order of micrometers and the periodic grooves in the order of nanometers are formed on the surface of the culture substrate of the present disclosure so as to be arranged in parallel. Therefore, the culture substrate of the present disclosure contributes to the induction of the differentiation of stem cells and promotes the induction of the differentiation of stem cells induced by a differentiation inducing factor, thus making it possible to efficiently induce the differentiation of stem cells into a desired type of cell.

For example, in the above-mentioned embodiments, in the culture substrate having a periodic fine structure in the order of micrometers and a periodic fine structure in the order of nanometers on the same surface where stem cells are to be cultured on the surface, the periodic fine structure in the order of micrometers is formed as periodic lattice grooves, and the periodic fine structure in the order of nanometers is formed as periodic projections.

With the present disclosure, a culture substrate for culturing stem cells can be provided in which the periodic fine structure in the order of micrometers is formed as periodic lattice grooves, and the periodic fine structure in the order of nanometers is formed as periodic projections. The periodic lattice grooves in the order of micrometers and the periodic projections in the order of nanometers are simultaneously formed on the surface of the culture substrate of the present disclosure. Since these two types of periodic structures coexist, the biocompatibility and the biological cell affinity can be further improved. Moreover, with the culture substrate of the present disclosure, stem cells can be efficiently proliferated in a state in which the undifferentiation property is maintained, thus making it possible to stably provide a high-quality stem cell population.

Furthermore, the culture substrate of the present disclosure promotes the induction of the differentiation of stem cells induced by a differentiation inducing factor, thus making it possible to efficiently induce the differentiation of stem cells into a desired type of cell, particularly nerve cells and adipocytes. Accordingly, the culture substrate of the present disclosure can contribute to the further development of techniques using stem cells such as those in regenerative medicine and drug development screening, which require a large amount of high-quality stem cells.

Furthermore, in the above-mentioned embodiments, the periodic lattice grooves in the order of micrometers have a width of 1 to 20 μm, a depth of 0.3 to 2 μm, and a pitch of 1 to 100 μm, and the periodic projections in the order of nanometers have a diameter of 0.1 to 1 μm, a height of 0.01 to 0.5 μm, and a pitch of 0.1 to 1 μm.

With the present disclosure, a culture substrate for culturing stem cells can be provided in which the periodic fine structure in the order of micrometers is formed as periodic lattice grooves and the periodic fine structure in the order of nanometers is formed as periodic projections, and the sizes of the two types of periodic fine structures are optimized. Since the periodic lattice grooves in the order of micrometers and the periodic projections in the order of nanometers with a favorable size are simultaneously formed on the surface of the culture substrate of the present disclosure, the biocompatibility and the biological cell affinity can be further improved, thus making it possible to efficiently induce the differentiation of stem cells into a desired type of cell, particularly nerve cells and adipocytes.

For example, in the above-mentioned embodiments, titanium is used as a material of the culture substrate having a periodic fine structure in the order of micrometers and a periodic fine structure in the order of nanometers on the same surface where stem cells are to be cultured on the surface.

With the present disclosure, the culture substrate can be produced by forming the periodic fine structure on a titanium material having high biocompatibility and biological cell affinity, and therefore, a further improvement in biocompatibility and biological cell affinity can be expected.

For example, in the above-mentioned embodiments, a method for producing a culture substrate is evoked that includes a step of forming the periodic grooves in the order of micrometers on a substrate surface through non-thermal cutting using an ultra-short pulse laser and a step of forming the periodic grooves in the order of nanometers on the substrate surface by forming a periodic nanostructure using linearly polarized light emitted by the ultra-short pulse laser.

With the present disclosure, a method for producing a culture substrate having a periodic fine structure in the order of micrometers and a periodic fine structure in the order of nanometers on the same surface where stem cells are to be cultured on this surface, can be provided, the periodic fine structure in the order of micrometers and the periodic fine structure in the order of nanometers in the culture substrate being formed as periodic grooves. The periodic fine structure can be formed in a simple manner by scanning the substrate surface using an ultra-short pulse laser, for example, which has an advantage that there is little restriction on the production because processing can be performed in atmospheric air due to little thermal influence, for example. Accordingly, the culture substrate of the present disclosure can be produced in a simple manner at low cost

For example, in the above-mentioned embodiments, a method for producing a culture substrate is evoked that includes a step of forming the periodic lattice grooves in the order of micrometers on a substrate surface through non-thermal cutting using an ultra-short pulse laser and a step of forming the periodic projections in the order of nanometers on the substrate surface by forming a periodic nanostructure using circularly polarized light emitted by the ultra-short pulse laser.

With the present disclosure, a method for producing a culture substrate having a periodic fine structure in the order of micrometers and a periodic fine structure in the order of nanometers on the same surface where stem cells are to be cultured on this surface, can be provided, the periodic fine structure in the order of micrometers being formed as periodic lattice grooves and the periodic fine structure in the order of nanometers being formed as periodic projections in the culture substrate. The periodic fine structure can be formed in a simple manner with scanning of the substrate surface using an ultra-short pulse laser, for example, which has an advantage that there is little restriction on the production because processing can be performed in atmospheric air due to little thermal influence, for example. Accordingly, the culture substrate of the present disclosure can be produced in a simple manner at low cost.

The principles, preferred embodiment and mode of operation of the present invention have been described in the foregoing specification. However, the invention which is intended to be protected is not to be construed as limited to the particular embodiments disclosed. Further, the embodiments described herein are to be regarded as illustrative rather than restrictive. Variations and changes may be made by others, and equivalents employed, without departing from the spirit of the present invention. Accordingly, it is expressly intended that all such variations, changes and equivalents which fall within the spirit and scope of the present invention as defined in the claims, be embraced thereby.

### REFERENCE SIGNS LIST

1 Culture substrate
2 Periodic fine structure in the order of micrometers (periodic microstructure)
2a Periodic grooves in the order of micrometers (periodic microgrooves)
2b Periodic lattice grooves in the order of micrometers (periodic lattice microgrooves)
3 Periodic fine structure in the order of nanometers (periodic nanostructure)
3a Periodic grooves in the order of nanometers (periodic nanogrooves)
3b Periodic projections in the order of nanometers (periodic nanoprojections)
4 Hybrid periodic fine structure (hybrid periodic structure)
4a Hybrid periodic grooves
4b Hybrid periodic lattice grooves+periodic projections

1. A culture substrate having a periodic fine structure in the order of micrometers and a periodic fine structure in the order of nanometers on the same surface where stem cells are to be cultured on the surface.

2. The culture substrate according to claim 1, wherein the periodic fine structure in the order of micrometers and the periodic fine structure in the order of nanometers are formed as periodic grooves.

3. The culture substrate according to claim 2, wherein the periodic grooves in the order of micrometers have a width of 1 to 20 μm, a depth of 0.3 to 2 μm, and a pitch of 1 to 100 μm, and the periodic grooves in the order of nanometers have a width of 0.1 to 1 μm, a depth of 0.01 to 0.5 μm, and a pitch of 0.1 to 1 μm.

4. The culture substrate according to claim 2, wherein the periodic grooves in the order of micrometers and the periodic grooves in the order of nanometers are arranged in parallel.

5. The culture substrate according to claim 1, wherein the periodic fine structure in the order of micrometers is formed as periodic lattice grooves, and the periodic fine structure in the order of nanometers is formed as periodic projections.

6. The culture substrate according to claim 5, wherein the periodic lattice grooves in the order of micrometers have a width of 1 to 20 μm, a depth of 0.3 to 2 μm, and a pitch of 1 to 100 μm, and the periodic projections in the order of nanometers have a diameter of 0.1 to 1 μm, a height of 0.01 to 0.5 μm, and a pitch of 0.1 to 1 μm.

7. The culture substrate according to claim 1, wherein titanium is used as a material of the culture substrate.

8. A method for producing the culture substrate according to claim 2, the method comprising:
a step of forming the periodic grooves in the order of micrometers on a substrate surface by non-thermal cutting using an ultra-short pulse laser; and
a step of forming the periodic grooves in the order of nanometers on the substrate surface by forming a periodic nanostructure using linearly polarized light emitted by the ultra-short pulse laser.

9. A method for producing the culture substrate according to claim 5, the method comprising:
a step of forming the periodic lattice grooves in the order of micrometers on a substrate surface by non-thermal cutting using an ultra-short pulse laser; and
a step of forming the periodic projections in the order of nanometers on the substrate surface by forming a periodic nanostructure using circularly polarized light emitted by the ultra-short pulse laser.
